(19) United States
(12) Patent Application Publication
(10) Pub. No.: US 2026/0159972 A1
(43) Pub. Date: Jun. 11, 2026

(54) HETEROCATALYSTS FOR HYDROGEN GENERATION AND CO2 CONVERSION BASED ON ELECTROCHEMICAL AND PHOTOCHEMICAL TECHNOLOGIES

(71) Applicant: Khalifa University of Science and Technology, Abu Dhabi (AE)

(72) Inventors: Maria Lourdes Vega Fernandez, Abu Dhabi (AE); Yuting Li, Abu Dhabi (AE); Muhammad Ashraf Sabri, Abu Dhabi (AE)

(21) Appl. No.: 19/408,227

(22) Filed: Dec. 3, 2025

Related U.S. Application Data
(60) Provisional application No. 63/728,444, filed on Dec. 5, 2024.

(51) Int. Cl. C25B 11/075; C25B 1/04; C25B 1/23; C25B 1/55; C25B 11/052; C25B 11/065

(52) U.S. Cl. CPC C25B 11/075; C25B 1/04; C25B 1/23; C25B 1/55; C25B 11/052; C25B 11/065; G16C 20/70

(57) ABSTRACT

Disclosed herein are heterocatalysts for hydrogen generation and carbon dioxide conversion based on electrochemical and photochemical technologies. The catalysts may be used for a hydrogen evolution reaction from water splitting and/or for a carbon dioxide reduction reaction. The catalysts may comprise metal sulfide. The catalysts may be identified using machine learning algorithms.

### CROSS-REFERENCES TO RELATED APPLICATIONS

This application claims the benefit of U.S. Provisional Patent Application No. 63/728,444, filed Dec. 5, 2024, the entire contents of which are hereby incorporated by reference for all purposes in its entirety.

### BACKGROUND OF THE INVENTION

The electrochemical reduction of carbon dioxide, CO₂, is referred to as a CO₂RR process. It is used to convert CO₂ into reduced chemical species using electrical energy. CO₂RR has been the subject of recent research and commercial interest because of environmental reasons, including its potential to reduce greenhouse gas emissions. The cost and energy required to run the process, however, remain high.

Water splitting for hydrogen and oxygen reduction is referred to as a HER process. It is used to decompose water into hydrogen and oxygen through various methods, including electrolysis, thermochemical cycles, and photochemical reactions. Like CO₂RR, it has been the subject of recent research and commercial interest for environmental reasons, including for clean hydrogen fuel production.

CO₂RR and HER are competing reactions and there is a need for catalysts that can efficiently drive these reactions. For the currently widely used photo-catalysts, such as TiO₂, g-C₃N₄, and CdS, extensive studies have been conducted at the lab scale. The photocatalytic materials of pristine TiO₂ has the disadvantages of low light absorption capacity and wide band gap⁶⁵. Also, the photocatalytic reaction on pristine g-C₃N₄ remains insufficient to meet the requirements of large-scale applications due to the quick recombination of electron-hole pairs and lower visible light absorption capacity over 460 nm. The rational fabrication of g-C₃N₄-based heterojunctions via combination with various nanoparticles still exhibit relatively inferior utilization of light and dampen the photocatalytic efficiency⁶⁶. Regarding to CdS, it tends to degrade under light irradiation, especially in aqueous environments, leading to a loss of catalytic activity over time⁶⁷.

For the currently widely used electro-catalysts, such as Cu, Au, Pt, Ni and MoS₂, extensive exploration has been carried out in CO₂RR and HER applications. However, obtaining a specific C₂+ product over Cu-based electrodes is a challenging task due to the divergent selectivity of Cu materials toward different fuels and chemicals and the fact that efficiency and selectivity decrease at high current densities. Suppression of competing HER on Cu-based catalyst is another challenging task during the CO₂RR in an aqueous solution⁶⁸. Even for the most prominent catalyst, Pt, the catalytic activity in alkaline medium is hindered by the sluggish water dissociation step, resulting in a reaction rate that is 2-3 orders of magnitude lower than that in acidic solution. Also, the high cost of noble metal catalysts is a significant barrier to large-scale application. Ni is widely used in industrial-scale water electrolysis, particularly in the HER through the electrolysis of water in alkaline environments. However, in the acidic environment of PEM electrolysis, nickel would corrode and lose its effectiveness, making it unsuitable for this specific application⁶⁹. The performance of MoS₂-based electrocatalysts is still far away from the benchmark performance with its lower electronic conductivity, inactive basal plane, limited edges sites, unexposed active site structures and instability⁷⁰.

### BRIEF SUMMARY OF THE INVENTION

Covered embodiments of the present disclosure are defined by the claims, not this summary. This summary is a high-level overview of various aspects of the invention and introduced some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification, any or all drawings and each claim.

The present disclosure is directed to new catalysts as well as new approaches to identifying such catalysts.

In some embodiments, the present disclosure is directed to a method of converting CO₂ to CO, the method comprising: photocatalytically converting CO₂ to CO in the presence of a catalyst, wherein the catalyst comprises a metal sulfide catalyst, wherein the selectivity toward CO is 90% or greater, and wherein distinct CO2-reduction activity has a Faradaic efficiency of at least 35%. In some aspects, the metal sulfide catalyst comprises Ni₃S₂, MnS₂, VS₄, V₃S₄, or combinations thereof. In some aspects, the metal sulfide catalyst comprises MnS₂. In some aspects, the metal sulfide catalyst comprises V₃S₄. The method may be conducted under a CO₂ pressure from 40 to 70 bar. The method may be conducted for a period of time from 30 minutes to 48 hours or from 1 hour to 48 hours. The method may be conducted with 5 to 100 mg catalyst. The metal sulfide catalyst may have a cathodic current density in an Ar-saturated blank (j_{Ar}) from −6.000 to −0.020 mA/cm². The metal sulfide catalyst may have a cathodic current density in a CO₂-saturated blank (j_{CO2}) from −2.000 to −0.030 mA/cm². A difference (Δj) between a CO₂-saturated blank (j_{CO2}) a cathodic current density in an Ar-saturated blank (j_{Ar}) may be from −0.020 to 4.000. The method may be conducted using a graphite sheet modified with the metal sulfide catalyst. The method may be conducted using an Ag/AgCl electrode.

In some embodiments, the present disclosure is directed to a method for predicting CO chemisorption on a surface using metal sulfide catalysts, the method comprising: inputting CO-surface distances, metal-sulfur bond lengths and energies above hull; predicting the CO chemisorption on a surface; setting a minimum acceptable chemisorption on a surface; and testing metal sulfide catalysts that meet the predicted minimum acceptable chemisorption. The predicting may be conducted by applying machine learning algorithms. In some aspects, at least two machine learning algorithms are used. In some aspects, four machine learning algorithms are used. In some aspects, Random Forest, trained with data from Density Functional Theory is used. In some aspects, three physical inputs are also input. The three physical inputs may be energy above the convex hull of bulk lattice, related to the stability, the metal-sulfur bond length, and distance between H and its 1^{st} neighbors on the top layer of the surface. The metal sulfide catalyst may have a selectivity toward CO is 90% or greater, and wherein distinct CO2-reduction activity has a Faradaic efficiency of at least 35%.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1 illustrates a flowchart of a machine-learning assisted screen network for metal sulfide catalysts for hydrogen production and CO₂ reduction in accordance with embodiments of the present disclosure.

FIG. 2 illustrates a periodic table highlighting metals involved in metal sulfide (MₓS_{y}) in accordance with embodiments of the present disclosure.

FIG. 3 illustrates a Pearson's linear correlation coefficients heat map in accordance with embodiments of the present disclosure.

FIGS. 4A-D illustrate data distribution and feature engineering for the distribution of calculated H and CO adsorption energies derived from DFT (FIG. 4A), a streamlined diagram for feature engineering (FIG. 4B), feature importance ranking for H adsorption energy prediction in RF model (FIG. 4C), and feature importance ranking for CO adsorption energy prediction in RF model (FIG. 4D) in accordance with embodiments of the present disclosure.

FIGS. 5A-D illustrate the performance of ML algorithms for classification and regression with FIG. 5A showing a comparison of ML classification accuracy among 4 algorithms, FIGS. 5B and 5C showing Random Forest Regression between ML predicted scaled H adsorption energies and DFT calculated energies on the training and testing sets, respectively, and FIG. 5D showing a 3D visualization of CO adsorption energy relative to M-S bond length and the distance between CO and the surface in accordance with embodiments of the present disclosure.

FIGS. 6A-F illustrate model optimizations with FIG. 6A showing the accuracy of the RF Classifier as a function of the ‘n_estimators’ hyperparameter, FIG. 5B showing a Random forest feature importance for structural stability classification, FIG. 6C showing a learning curve for H adsorption energy regression with 5-fold Cross-Validation, FIG. 6D showing a learning curve for CO adsorption energy regression with 5-fold Cross-Validation, and FIGS. 6E and 6F showing the structure of Zr₅S₈ with different H adsorption sites before DFT structure optimization in accordance with embodiments of the present disclosure.

FIG. 7 illustrates a hyperparameter tuning process of RFR model for H adsorption energy prediction in accordance with embodiments of the present disclosure.

FIGS. 8A-F illustrate prediction performance of 3 ML algorithms on H adsorption energy in accordance with embodiments of the present disclosure.

FIGS. 9A-H illustrate prediction performance of 3 ML algorithms on CO adsorption energy in accordance with embodiments of the present disclosure.

FIGS. 10A-E illustrate an interpretation of ML with respect of distribution of H adsorption energy varies with d_H_1 less than 0.7 Å. (FIG. 10A), the distribution of H adsorption energy varies with average M-S bond length (FIG. 10B), the structure of LuS₂ before DFT optimization (FIG. 10C), the graphic structure of LuS₂ after DFT optimization (FIG. 10D), and the distribution of H adsorption energy varies with space group types (FIG. 10E) in accordance with embodiments of the present disclosure.

FIGS. 11A-B illustrate period performance of an RFR algorithm on CO adsorption energy using only one “sis_col” feature in accordance with embodiments of the present disclosure.

FIGS. 12A-C illustrate the distribution of ML-predicted H (FIG. 12A) and CO adsorption energies by trained RFR model (FIG. 12B) with the distribution of screened materials for HER (marked with circles) and CO₂RR (marked with stars) within the periodic table (FIG. 12C) in accordance with embodiments of the present disclosure.

FIGS. 13A-B illustrate schematic diagrams of the In₅S₄ bulk lattice structure (FIG. 13A) and a schematic diagram of the In₅S₄ surface structure. (FIG. 13B) in accordance with embodiments of the present disclosure.

FIGS. 14A-F illustrate CV curves of transition-metal sulfides in CO₂- and Ar-saturated 0.25 M NaHCO₃ at 20 mV·s⁻¹. CV curves are shown for (FIG. 14A) CdS, (FIG. 14B) CuS, (FIG. 14C) MoS₂, (FIG. 14D) Ni₃S₂, (FIG. 14E) MnS₂ and (14F) V₃S₄ in accordance with embodiments of the present invention.

FIG. 15 provides a comparison of CO₂-saturated CV for the CO₂-active sulfide catalysts V₃S₄ and MnS₂ in CO₂-saturated 0.25 M NaHCO₃ at a scan rate of 20 mV·s⁻¹ in accordance with embodiments of the present invention.

FIG. 16 shows photocatalytic CO₂-to-CO conversion over VS₄ and MnS₂ under high-pressure CO₂ in accordance with embodiments of the present invention.

### DETAILED DESCRIPTION OF THE INVENTION

### Introduction

The present disclosure is directed to new catalysts as well as new approaches to identifying such catalysts. The new catalysts may be used in a CO₂RR reaction for converting CO₂ to CO. The method may comprise photocatalytically converting CO₂ to CO in the presence of a catalyst, wherein the catalyst comprises a metal sulfide catalyst, wherein the selectivity toward CO is 90% or greater, and wherein distinct CO2-reduction activity has a Faradaic efficiency of at least 35%.

The present disclosure is also directed to a method for predicting CO chemisorption on a surface using metal sulfide catalysts. The method may comprise inputting CO-surface distances, metal-sulfur bond lengths and energies above hull; predicting the CO chemisorption on a surface; and testing metal sulfide catalysts that meet the predicted minimum acceptable chemisorption. By applying four Machine Learning algorithms, it was found that Random Forest, trained with data from Density Functional Theory (DFT), showed the best performance in predicting stability and adsorption energies, identifying 38 and 77 potential metal sulfides for HER and CO₂RR, respectively. Surprisingly, it was found that just three physical inputs (CO-surface distances, metal-sulfur bond lengths and energies above hull) could roughly forecast the CO chemisorption on surfaces.

Illustrative examples are given to introduce the reader to the general subject matter discussed herein and are not intended to limit the scope of the disclosed concepts. The following sections describe various additional features and examples with reference to the drawings in which like numerals indicate like elements, and directional descriptions are used to describe the illustrative aspects, but, like the illustrative aspects, should not be used to limit the present disclosure.

Before the present disclosure is described in detail, it is to be understood that the terminology used herein is for purposes of describing particular examples and embodiments only, and is not intended to be limiting.

In this specification and in the claims that follow, reference will be made to a number of terms that shall be defined to have the following meanings:

### Definitions

Articles “a” and “an” are used herein to refer to one or to more than one (i.e., at least one) of the grammatical object of the article. By way of example, “an element” means at least one element and can include more than one element.

As used herein, the terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

The terms “about” and “approximately” as used herein shall generally mean an acceptable degree of error for the quantity measured given the nature or precision of the measurements. Exemplary degrees of error are within 20% (%); preferably, within 10%; and more preferably, within 5% of a given value or range of values. Any reference to “about X” or “approximately X” specifically indicates at least the values X, 0.95X, 0.96X, 0.97X, 0.98X, 0.99X, 1.01X, 1.02X, 1.03X, 1.04X, and 1.05X. Thus, expressions “about X” or “approximately X are intended to teach and provide written support for a claim limitation of, for example, “0.98X.” Numerical quantities given herein are approximate unless stated otherwise, meaning that the term “about” or “approximately” can be inferred when not expressly stated. When “about” is applied to the beginning of a numerical range, it applies to both ends of the range.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition, in a description of a method, or in a description of elements of a device, is understood to encompass those compositions, methods, or devices consisting essentially of and consisting of the recited components or elements, optionally in addition to other components or elements. The invention illustratively described herein suitably may be practiced in the absence of any element, elements, limitation, or limitations which is not specifically disclosed herein.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

Specific details are given in the description to provide a thorough understanding of the embodiments. However, it will be understood that the embodiments may be practiced without these specific details. For example, specific computational models, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

### Machine Learning

Integrating machine learning (ML) density functional theory (DFT) allows the reliable screening of efficient metal sulfide heterocatalysts for hydrogen evolution reaction (HER) and CO₂ reduction reaction (CO₂RR). Among the four studied ML algorithms, Random Forest, trained with data from DFT, demonstrated the best performance in predicting stability and adsorption energies, identifying 38 and 77 potential metal sulfides for HER and CO₂RR, respectively. When adding electron transfer capacity and cost, CdS, PbS, ZnS, MoS₂ and SnS₂ have been identified as the most efficient and cost-effective options for HER while FeS₂, CdS, MnS₂, PbS₂, ZnS, TiS₂ and CuS apply for CO₂RR. Remarkably, just three physical inputs (CO-surface distances, metal-sulfur bond lengths and energies above hull) were able to roughly forecast the CO chemisorption on surfaces. The present disclosure quantitatively correlates catalyst performance with fundamental properties to guide the rational design of catalysts, highlights primary challenges in aligning ML predictions with DFT, and identifies promising affordable catalysts for HER and CO₂RR to expand the catalyst database and advance the materials science for renewable applications.

Currently, electrochemical^{0,0} and photochemical^{0,0} technologies emerge as the most promising approaches for CO₂RR and HER from H₂O splitting, offering potential solutions for mitigating global warming and promoting a sustainable energy future. Advances in heterogeneous catalyst design have significantly enhanced efficiency and scalability in these technologies. In particular, there are numerous research endeavors based on DFT calculations aiming at providing accurate predictions of geometric and electronic properties based on quantum mechanics, focused on understanding how small molecules interact with solid surfaces⁰⁻⁰. Another area of unequivocal expansion in materials science is related to the development of Machine Learning (ML) techniques⁰⁻⁰. ML combined with highly-accurate quantum mechanical methods has the ability to accurately extract features embedded in the input data and to recognize the objective behaviors, hence enabling a variety of predictions such as estimating DFT-computed properties⁰, surface-adsorbate interactions^{0,0}, chemical reactions^{0,0} and catalysts discovery and design⁰, among others⁰. Specific to this disclosure, some comprehensive reviews in the literature⁰⁻⁰ highlighted the applications of ML to screen materials for HER or CO₂RR, and summarized available materials datasets that can be used to explore catalysts, including open datasets such as Materials Project⁰, Open Quantum Materials Database (OQMD⁰), Catalysis-hub.org⁰, among others.

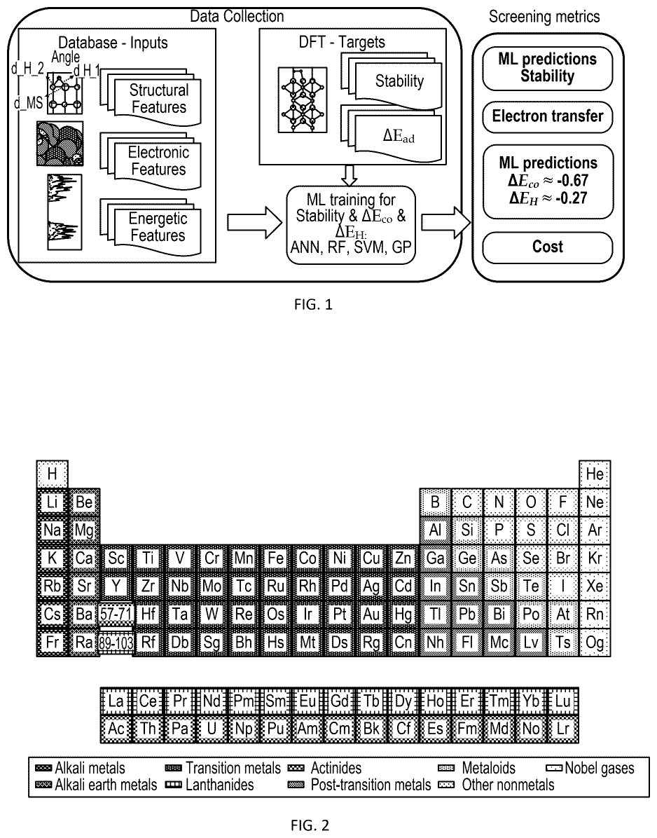

Particularly, metal sulfides have gained attention as promising catalysts for both HER and CO₂RR due to their unique electronic and optical properties, such as high charge transfer efficiency, narrower band gaps and abundant active sites, leading to enhanced catalytic activity⁰⁻⁰. Compared to metal oxides, the valence bands of metal sulfides are primarily occupied by sulfur 3p orbitals with higher atomic orbital energies than oxygen, leading to shallow valence bands and narrow bandgaps that enable effective utilization as photocatalysts over a broader range of visible light wavelengths^{0,0}. Hence, to identify the most promising candidates, the present disclosure describes a screening process that combines several key descriptors to represent structural stability, charge transfer capacity and catalytic activity. Given the large number of possible metal sulfide compositions, facets and active sites, it is challenging to access all these descriptor values through experimental or DFT calculations alone. Especially, intermediate adsorption energies on the metal sulfide surfaces are not readily available in online databases. Therefore, the proposed and implemented ML-aided screening approach explores optimal metal sulfide catalysts over 887 MₓS_{y} lattices as shown in FIG. 1.

The present disclosure also focuses on proposing novel heterocatalysts for hydrogen evolution reaction (HER) from water splitting and CO₂ reduction reaction (CO₂RR) to advance clean energy technologies and reduce greenhouse gas emissions. In hydrogen generation, these catalysts facilitate the splitting of water into hydrogen and oxygen using either electrochemical or photochemical methods. The produced hydrogen serves as a clean fuel, essential for applications like fuel cells, but also for other industrial processes. For CO₂ conversion, these catalysts help transforming carbon dioxide into valuable chemicals and fuels, such as methanol and hydrocarbons among others, through electrochemical and photochemical processes. The present disclosure is also related to the procedure in which these materials were selected, combining Machine Learning Techniques with Density Functional Theory and detecting the most relevant features of these materials for their desired application.

Platinum is considered the benchmark catalyst for electro-catalytic HER due to its excellent catalytic activity and low overpotentials in acidic solutions. However, the main issue with platinum is its high cost and limited availability, which makes it impractical for large-scale applications. Copper is one of the most studied catalysts for electro-catalytic CO₂RR due to its unique ability to produce hydrocarbons like methane and ethylene from CO₂, but it often suffers from low selectivity and efficiency at high current densities. Photocatalysts for these two applications are mostly in the research and laboratory phase due to their poor long-term stability, charge separation and catalytic selectivity, limited quantum efficiency to absorb visible light with wide bandgaps, high cost or environmental toxicity.

Transition metal sulfides (TMS) have shown promising HER and CO₂RR activity, due to their unique electronic and optical properties, such as high charge transfer efficiency, narrower band gaps and abundant active sites, leading to enhanced catalytic activity, particularly in acidic and neutral environments. Compared to metal oxides, the valence bands of metal sulfides are primarily occupied by sulfur 3p orbitals with higher atomic orbital energies than oxygen, leading to shallow valence bands and narrow bandgaps. Therefore, this invention will propose several novel catalysts which are designed to achieve good stability, charge transfer capacity and catalytic activity, making them crucial for sustainable energy production.

Machine Learning (ML) combined with Density Functional Theory (DFT) approaches have been applied to screen 881 TMS materials available at the open source Material Project database to identify the suitable compositions and lattice structures for catalytic HER and CO₂RR, presenting a significant advancement in catalyst discovery. Among these, several novel materials have been successfully synthesized in experiments for other applications, while a few remain theoretical structures that have yet to be synthesized experimentally.

A summary of the proposed catalytic materials for HER is provided in Table 1, listing the specific TMS screened for their potential in HER, their crystal structure, details the electrical conductivity, bandgaps that influence the efficiency of electron transfer during reactions and light utilization to initial reactions, the intermediate (H) adsorption energies (E_{H}) which significantly impacts the overall catalytic efficiency in photocatalysis, overpotential in electrocatalysis, the cost of synthesizing materials, and whether the material has been studied and experimentally synthesized for other applications or only theoretically predicted. Similarly, A summary of the proposed catalytic materials for CO₂RR is provided in Table 2, listing the specific TMS screened for their potential in CO₂RR, their crystal structure, details the electrical conductivity, bandgaps that influence the efficiency of electron transfer during reactions and light utilization to initial reactions, the intermediate (CO) adsorption energies (E_{CO}) which significantly impacts the overall catalytic efficiency in photocatalysis, overpotential in electrocatalysis, the cost of synthesizing materials, and whether the material has been studied and experimentally synthesized for other applications or theoretically predicted but not synthesized yet.

The optimal adsorption energy in Table 1 and Table 2 indicates an ideal balance between reactant adsorption resistance and the ease of proton detachment or products desorption from the catalyst surface. The relationship revealed that an optimal activity and selectivity can be achieved with a EH of −0.27 eV and ECO of −0.67 eV. The physical understanding behind these values is that for HER, elements that can easily adsorb and desorb H are crucial, while for CO2RR, elements that can stabilize the intermediate species of CO2 reduction are important. Therefore, all the potential materials listed in Tables 1 and 2 have active adsorption sites with EH around −0.27 eV (e.g., from −1.00 to 0.01 eV) and ECO around −0.67 eV (e.g., from −1.25 to 0.3 eV). All materials listed in this table are stable.

### Methods for Converting CO₂ to CO

As described herein, the present disclosure is also directed to new catalyst, including those described in the tables above and below, and to methods of using the catalysts in a CO₂RR method to convert CO₂ to CO. The catalyst comprises a metal sulfide catalyst that is able to achieve s selectivity toward CO of 900% or greater, e.g., at least 920%, at least 9400, at least 96%, up to 10000 or 99.900 The catalyst may also have a distinct CO2-reduction activity Faradaic efficiency of at least 35%, e.g., at least 37%, at least 40%, at least 41%, or at least 43%, from 35 to 500%, from 37 to 500%, from 37 to 4500 from 40 to 500%, or from 40 to 4500

The metal sulfide catalyst may comprise Ni₃S₂, MnS₂, VS₄, V₃ S₄, or combinations thereof. For example, the metal sulfide catalyst may be Ni₃S₂, MnS₂, VS₄, or V₃ S₄. The catalyst may be incorporated into a graphite sheet. The catalyst may be included in the method in an amount from 5 to 100 mg, e.g., from 10 to 100 mg, from 25 to 100 mg, from 45 to 100 mg, from 5 to 75 mg, from 10 to 75 mg, from 25 to 75 mg, from 45 to 75 mg, from 5 to 50 mg, from 10 to 50 mg, from 25 to 50 mg, from 50 to 100 mg, from 50 to 75 mg, or from 75 to 100 mg.

The method may be conducted under a CO₂ pressure from 40 to 70 bar, e.g., from 40 to 60 bar, from 40 to 50 bar, from 50 to 70 bar, from 50 to 60 bar, or from 60 to 70 bar.

The method may be conducted for a period of time from 30 minutes to 48 hours, e.g., from 1 to 48 hours, from 5 to 48 hours, from 10 to 48 hours from 15 to 48 hours, from 20 to 48 hours, from 30 to 48 hours, from 40 to 48 hours, from 30 minutes to 40 hours, from 1 to 40 hours, from 5 to 40 hours, from 10 to 40 hours, from 12 to 40 hours, from 15 to 40 hours, from 20 to 40 hours, from 30 to 40 hours, from 30 minutes to 30 hours, from 1 to 30 hours, from 5 to 30 hours, from 10 to 30 hours, from 12 to 30 hours, from 15 to 30 hours, from 20 to 30 hours, from 30 minutes to 20 hours, from 1 to 20 hours, from 5 to 20 hours, from 10 to 20 hours, from 12 to 20 hours, from 15 to 20 hours, 30 minutes to 15 hours, from 1 to 15 hours, from 5 to 15 hours, from 10 to 15 hours, or from 12 to 15 hours. In some aspects, the period of time may be approximately 24 hours.

The metal sulfide catalyst may have a cathodic current density in an Ar-saturated blank (j_{Ar}) from −6.000 to −0.020 mA/cm², e.g., from −5.500 to −0.025 mA/cm².

The metal sulfide catalyst may have a cathodic current density in a CO₂-saturated blank (j_{CO2}) from −2.000 to −0.030 mA/cm², e.g., from −1.500 to −0.035 mA/cm²

The difference (Δj) between a CO₂-saturated blank (j_{CO2}) a cathodic current density in an Ar-saturated blank (j_{Ar}) may be from −0.020 to 4.000, e.g., from −0.025 to 3.500 mA/cm².

The method may be conducted using an Ag/AgCl electrode.

In the preceding description, various embodiments have been described. For purposes of explanation, specific configurations and details have been set forth in order to provide a thorough understanding of the embodiments. However, it will also be apparent to one skilled in the art that the embodiments may be practiced without the specific details. Furthermore, well-known features may have been omitted or simplified in order not to obscure the embodiment being described. A person of skill in the art may understand that steps described above may be omitted, may be performed in a different order, or may include repeating steps described herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### EXAMPLES

The following examples are offered to illustrate, but not to limit, the present disclosure.

### Example 1

FIG. 1 provides an illustration of a flowchart of the machine learning-assisted screening network for metal sulfide catalysts for hydrogen production and CO₂ reduction. The process involves three main steps, from left to right: 1. Data collection from various online databases to serve as input features for ML model, followed by DFT calculations to obtain adsorption energies (E_{ad}) as ML targets; 2. ML models: constructing ML models for both classifications of structural stability and regression of adsorption energies, four different supervised ML algorithms were selected: the most common type of artificial neural network (ANN) with Multilayer Perceptron (MLP), random forest (RF), support vector machine (SVM), and gaussian process (GP); 3. Large-scale screening using four descriptors to determine the catalytic performance for HER and CO₂RR: stability, electron transfer (band gap), intermediate adsorption energy for catalytic activity and synthesize cost. DFT was introduced as a computational tool to investigate the convergence capacity of over 3000 adsorbed structure optimizations and to calculate the H and CO adsorption energies on these surfaces, serving as targets for supervised ML models.

FIG. 2 provides a periodic table highlighting metals involved in Metal sulfide (MₓS_{y}). The elements studied in this work are highlighted in black font, while the unstudied elements are depicted in grey. FIG. 2 highlights the range of metal elements encompassed within these 887 MₓS_{y} lattices, representing the entirety of MₓS_{y} lattices available in the Materials Project database. The developed ML models successfully and rapidly classify stabilities and predicted adsorption energies on 2274 unique metal sulfide facets. Throughout the ML process, physical knowledge was incorporated into large scale data learning by analyzing the important descriptors most related to catalytic performance, further revealing the nature of chemical adsorption and opening up new avenues for ML discovery of novel materials with desired catalytic properties.

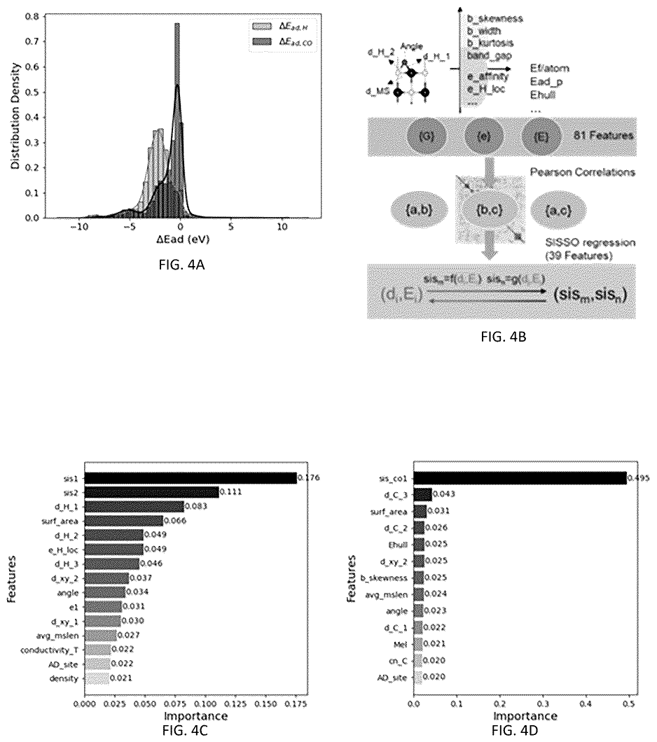

The essence of supervised ML lies in its ability to discern complex patterns within the input data, enabling the prediction of desired properties for novel materials. After data preprocessing, a distribution of calculated H and CO adsorption energies is shown FIG. 4A, which primarily falls between −3 and 0 eV. By integrating structural descriptors, electronic features, and energy-related attributes as inputs (see FIGS. 4A-D), the model adeptly forecasts the adsorption energies and stability of uncharted materials. FIGS. 4A-D shows Data distribution and feature engineering. FIG. 4A illustrates the distribution of calculated H and CO adsorption energies derived from DFT. FIG. 4B illustrates a streamlined diagram for feature engineering. Starting with a set of 81 features, which can be categorized into geometrical (G), electronic (e), and energetic (E) features. Then calculations of Pearson correlation coefficients were performed to eliminate features with a correlation of higher than 0.75, indicating high redundancy. Only one effective parameter was retained for further modelling. Subsequently, the Sure Independence Screening and Sparsifying Operator (SISSO) technique was applied to obtain highly effective features (i.e., sis1, sis2, sis_col, see Eq 4-6). FIG. 4C illustrates feature importance ranking for H adsorption energy prediction in RF model. Features were removed when an importance score is less than 0.02. FIG. 4D illustrates feature importance ranking for CO adsorption energy prediction in RF model. Features were removed if an importance score was less than 0.02. The sis_co1 value is involving structural and energetic factors: a distance term, an average bond length in the MₓS_{y} lattice and the energy above the convex hull, related to the stability. This metric can capture the interplay between structural and energetic properties influencing the CO adsorptions.

The principle behind the feature selection in this work is to utilize easily accessible information, primarily derived from structures or online databases, to harness fundamental physical properties for the prediction of adsorption energies and gain insight into chemisorption mechanisms. The selected features and their abbreviations are listed in Table 3. A good sample dataset and feature selection significantly improved the performance of ML models, especially in the material design field with very few and limited data.

TABLE 3
Proposed initial features for the ML training dataset. These features mainly
obtained from the crystal structure (C), pseudopotential files in VASP¹, Pymatgen² (P),
Materials Project³ (MP) and Mendeleev⁴ (M) database.

| Type | Feature | Definition | Source |
|---|---|---|---|
| Geometric | 1Ele | 1^{st} atom in H/CO closet neighbor list | C |
| | 2Ele | 2^{nd} atom in H/CO closet neighbor list | C |
| | 3Ele | 3^{rd} atom in H/CO closet neighbor list | C |
| | AD_site | Adsorption site defined by pymatgen | P |
| | angle | Angle between adsorbate and surface | C |
| | atomic_M | Atomic mass of metal | M |
| | atomic_n | Atomic number of metal | M |
| | atomic_r | Atomic radius of metal | M |
| | avg_mslen | Average metal-sulfur (M—S) bond length | C |
| | cn_H | Coordination number for H/CO within 3 Å | C |
| | covalent_r | Covalent radius of metal | M |
| | d_H/C_1 | Distance between H/CO and its 1^{st} neighbor on the | C |
| | | surface | |
| | d_H/C_2 | Distance between H/CO and its 2^{nd} neighbor on the | C |
| | | surface | |
| | d_H/C_3 | Distance between H/CO and its 3^{rd} neighbor on the | C |
| | | surface | |
| | d_xy_1 | Distance between H/CO and its 1^{st} neighbor from xy | C |
| | | direction | |
| | d_xy_2 | Distance between H/CO and its 2^{nd} neighbor from xy | C |
| | | direction | |
| | facet | Surface facet | P |
| | metallic_r | Metallic radius of the metal atom | M |
| | n_atoms_top | Number of atoms in the top layer of surface | C |
| | n_metal | Number of metal atoms | C |
| | n_xy_1 | Number of atoms at distance d_xy_1 from H/CO | C |
| | n_xy_2 | Number of atoms at distance d_xy_2 from H/CO | C |
| | nsites | Number of atoms in the lattice | C |
| | Space_Group | Space group of bulk lattice | C |
| | surf_area | Surface area | C |
| | V | Volume of bulk lattice | C |
| | vdw_r | van der Waals radius of metal | M |
| Electronic | b_center | Band center for bulk lattice | MP |
| | b_filling | Band filling for bulk lattice | MP |
| | b_kurtosis | Band kurtosis for bulk lattice | MP |
| | b_skewness | Band skewness for bulk lattice | MP |
| | b_width | Band width for bulk lattice | MP |
| | band_gap | Band gap for bulk lattice | MP |
| | block_N | The block number of metal | M |
| | cbm | Conduction band minimum of bulk lattice | MP |
| | d_orb | Quantum number of the d orbital from metal | M |
| | dipole_polar | Polarizability of the dipole moment | M |
| | e_affinity | Electron affinity | M |
| | efermi | Fermi energy of band structure | MP |
| | Ele_Allen | Allen electronegativity | M |
| | Ele_Pauling | Pauling electronegativity | M |
| | e1 | Pauling electronegativity of 1Ele | M |
| | e2 | Pauling electronegativity of 2Ele | M |
| | e3 | Pauling electronegativity of 3Ele | M |
| | group_id | Group number of metal element in periodic table | M |
| | e_H/CO_loc | Local electronegativity around H/CO | M |
| | N_d_orb | Number of electrons in the d orbital of metal | M |
| | N_p_orb | Number of electrons in the p orbital of metal | M |
| | N_s_orb | Number of electrons in the s orbital of metal | M |
| | p_orb | Quantum number of the p orbital from metal | M |
| | period_id | Period of metal element in periodic table | M |
| | s_orb | Quantum number of the s orbital from metal | M |
| | total_electrons | Number of electrons in the surface | MP |
| | vbm | Valence band maximum | MP |
| Energetic | E/atom | Total energy per atom of bulk lattice | MP |
| | Ead_pCO | Adsorption energy of CO on pure metal lattice | Our |
| | | | DFT |
| | Ead_pH | Adsorption energy of H on pure metal lattice | Our |
| | | | DFT |
| | EAUG | Augmented basis in pseudopotentials | VASP |
| | e_pot | Potential energy of an electron in the pseudopotential | VASP |
| | Ef/atom | Formation energy per atom of bulk lattice | MP |
| | Ehull | Energy above the convex hull of bulk lattice | MP |
| | Eion | Ionization energy of metal | MP |
| | evapor_h | Heat of vaporization | MP |
| | fusion_heat | Heat of fusion | MP |
| | h_cap | Heat capacity of metal | MP |
| | hf | Heat of formation | MP |
| | ICORE | Pseudopotential setting of metal | MP |
| | RCORE | Pseudopotential setting of metal | VASP |
| | RDEP | Pseudopotential setting of metal | VASP |
| | RDEPT | Pseudopotential setting of metal | VASP |
| | RMAX | Pseudopotential setting of metal | VASP |
| | RPACOR | Pseudopotential setting of metal | VASP |
| | RWIGS | Pseudopotential setting of metal | VASP |
| | Un_E/atom | Uncorrected total energy per atom of bulk lattice | MP |
| Other | Boil | Boiling point of metal | M |
| | density | Density of the bulk lattice | MP |
| | liquid_range | Liquid range of metal | M |
| | mag | Total magnetization of bulk lattice | MP |
| | Mel | Melting point of metal | M |
| | n | Refractive index | MP |
| | conductivity_T | Thermal conductivity of metal | M |

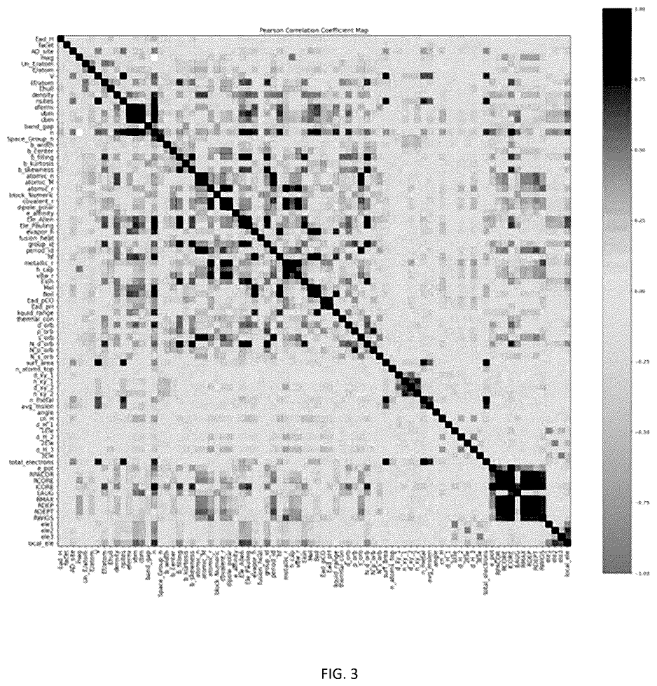

To address the presence of multiple collinearities and reduce high-dimensional features the Pearson correlation coefficients⁰ were calculated (see FIG. 3). FIG. 3 shows a heat map which was structured with the target variable positioned in the first column, followed by 81 additional features. Each cell within the heat map represents the correlation coefficient between pairs of features, color-coded to indicate the strength and direction of the correlation. Sure Independence Screening and Sparsifying Operator (SISSO)⁰ was used to create new comprehensive features as shown in FIG. 4A. As seen in FIGS. 4C and 4D, the top two features with the highest importance scores are SISSO generated features, underscoring their substantial impact on the predictive performance of the model. Other features with high importance scores were mostly related to the adsorption structures, specifically the distance of nearest neighboring atoms of adsorbates (d_H/C_i, d_xy_i), which corresponds to the atomic interactions or attractive strength between adsorbate and catalyst surface, making them critical factors for determining the adsorption strength of the material.

Electronic features such as the local electronegativity of metal atoms within the first neighboring shell (e_H/C_loc) also play critical roles during the adsorption processes. Fundamentally, electronegativity reflects the attraction ability of an atom to electrons, and the augment of electronegativity clearly enhances an atom's attraction to electrons in the adjacent layer⁰. Additionally, the d-band skewness (d_skewness) and d-band width (d_width) of the MₓS_{y} bulk system which roughly correlated to the position of the resultant antibonding states formed from the surface-adsorbate interaction⁰. Moreover, the 4^{th} important feature for CO adsorption energy prediction is the energy above the convex hull (Ehull). This is because the convex hull represents the thermodynamically stable phase of a material at a given composition and pressure. Therefore, by comparing the importance of features, the most influential factors affecting catalytic performance can be identified, and the optimization and development of more effective catalysts can be guided.

### Evaluation of ML Algorithms Performance

The evaluation of different ML algorithms performances is crucial for determining the effectiveness and reliability of ML models to accurately predict outcomes on unseen data. Here, the performance of these four ML algorithms (i.e., RF, SVM, GP and MLP) was evaluated to predict the stabilities of H/CO adsorption structures and adsorption energies of H/CO on metal sulfides catalysts.

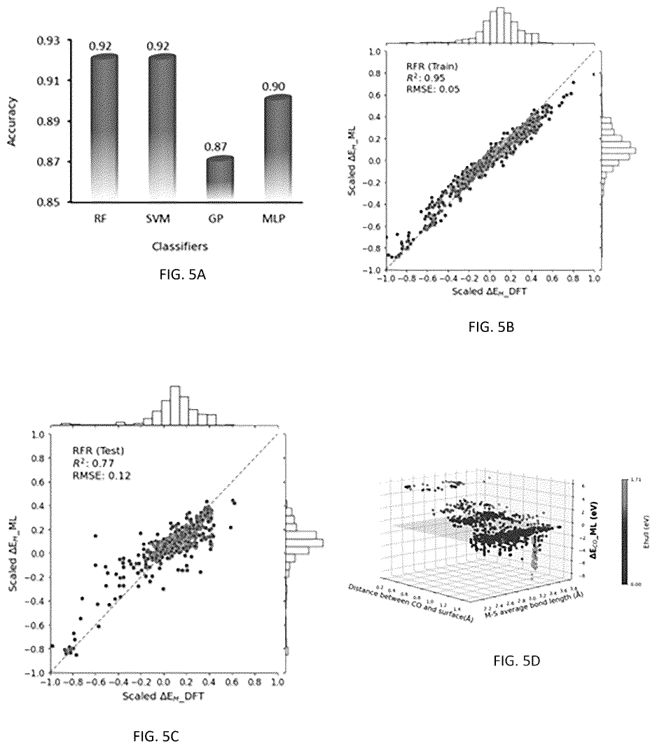

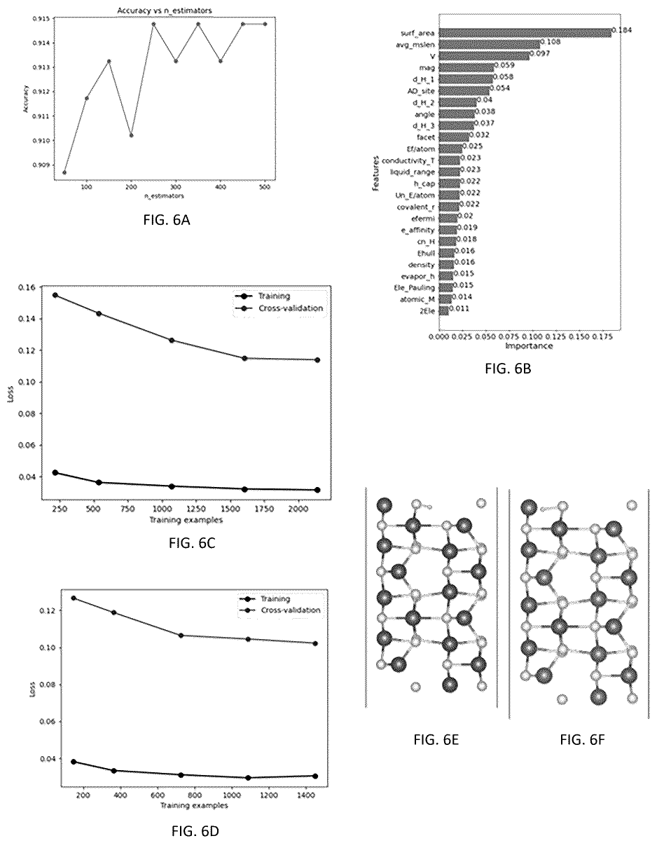

Applying ML to predict the convergence capacity of DFT simulations is necessary because if surfaces have poor initial guesses of electron density or too complex energy landscapes with multiple competing states, they might exhibit convergence difficulties and fail to reach the global minimum energy state. Thus, 4 ML classifiers were applied to preemptively classify the likelihood of convergence during structure optimization. Hyperparameter optimization was conducted for each classifier by systematically fine-tuning the model's hyperparameters (see FIG. 6A) and exploring different hyperparameter combinations, then the most suitable hyperparameters were selected to ensure the effectiveness and accuracy of the classifiers. The evaluation of each classifier's performance was conducted using accuracy metrics as shown in FIG. 5A, where it was observed that the MLP and RF demonstrated similar effectiveness—both achieving an accuracy of 0.92 (Eq.1), suggesting these two classifiers are reliable for capturing the DFT convergence behavior. From the feature importance ranking for RF classifier depicted in FIG. 6B, the geometric features have the upmost significance, including the surface area (surf_area), the average bond length between the metal and sulfur atoms (avg_mslen), lattice volume (v), etc. Different from the task of predicting adsorption energies, the determination of a structure convergence involves considering formation energy and heat capacity as pivotal factors. As these features are intimately related to thermodynamic responses under variable temperature conditions and energetic favorability, they are serving as critical indicators in the pre-selection of candidates.

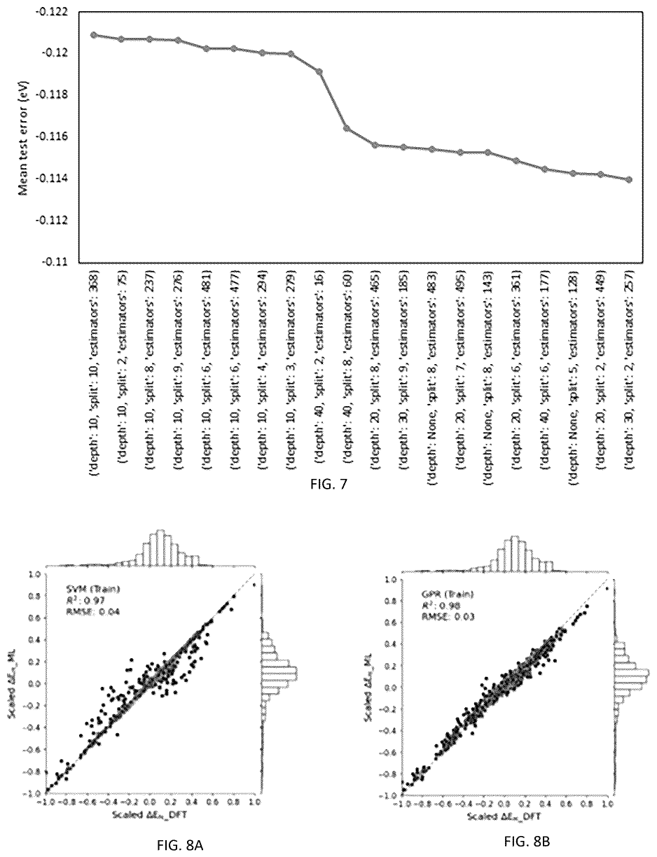

After classifying all the H/CO adsorption structures into convergence “true” or “false”, four ML regression models were further developed for the prediction of intermediate H/CO adsorption energies with multi-dimensional feature space. The target variable to train these supervised ML models was the adsorption energy derived from DFT, which serves as a critical parameter in understanding the adsorption process and determining the catalytic activity. The size of the training set also has a significant impact on the ML model's ability to learn and generalize. Adequate data ensures that the model has learned enough to have predictive power but has not started to memorize the training data to loss its generalization ability. The learning curve in FIG. 6C displays the training and cross-validation error of Random Forest regression (RFR) model for H adsorption energy prediction as a function of the training dataset size. By analyzing the learning curve, we can identify an optimal training dataset size of around 2,000 to achieve the desired performance. Also, in FIG. 6D, the cross-validation error continues to decrease with the increase of training samples before reaching 1,000 data size, which indicates that the model is learning from the training data and improving its ability to generalize to unseen data. But beyond 1,200 training examples, the cross-validation error began to slightly reduce. After identifying the appropriate training set size, hyperparameter tuning was then performed to systematically search for the optimal set of hyperparameters that yields the best performance (as shown in FIG. 7). There is a trend of decreasing in the cross-validation error during the hyperparameter tuning process, indicating an improvement in predictive accuracy as the depth of RFR model increases, then the trend followed by a plateau, suggesting an optimal depth has been reached.

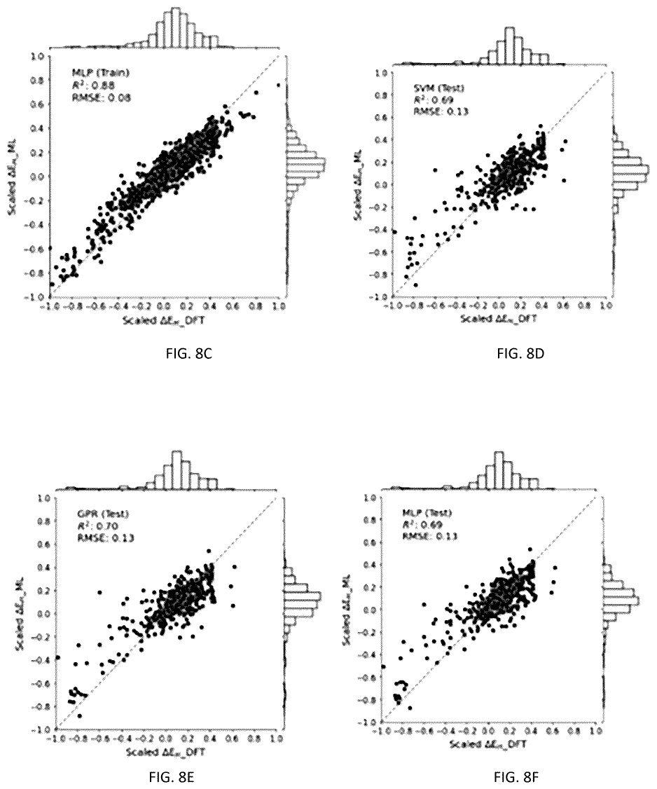

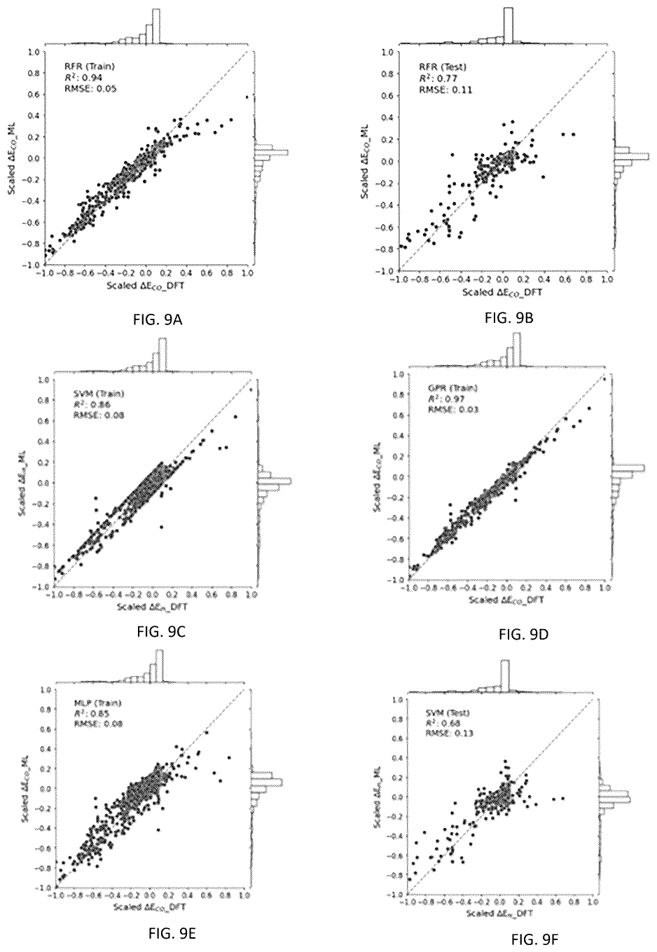

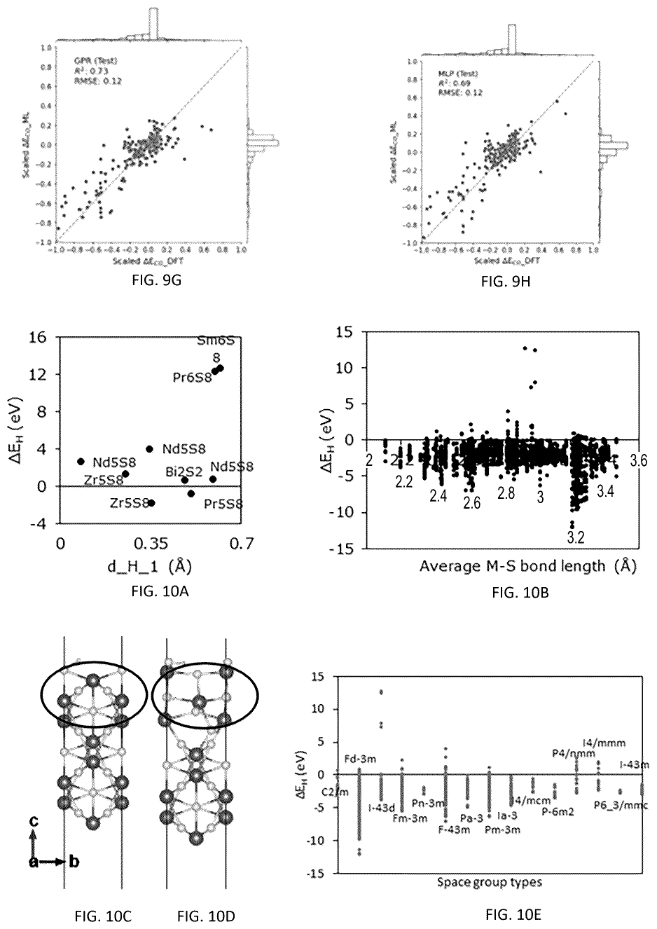

The ML performances for predicting H and CO adsorption energies are graphically represented in FIGS. 5B and 5C, FIGS. 8A-F, and FIGS. 9A-H. It can be inferred that RFR, being good at handling categorical problems, has the best predictive ability with the highest R² for the tested dataset among both H and CO adsorption cases, followed by Gaussian Process Regression (GPR). Regarding to the feature effects, as shown in FIG. 10A, when the neighbor distance (d_H 1) is less than 0.7 Å, the adsorption energy exhibits a high possibility to obtain large positive values, because atoms in very close proximity can produce significant electron cloud repulsive interactions. The average metal-sulfur bond length (avg_mslen) is also significantly correlated with the H adsorption energy, as depicted FIG. 10B, showing a high proportion of extremely large negative ΔE_{H} obtained when the bond length is around 3.2 Å. Specifically, these extremely negative values indicate a substantial rearrangement of surface atoms during DFT structural optimization, especially at the top layer, as illustrated in FIGS. 10C-D. This is referred to as “differential reconstruction,” where the surface reconstructs differently for the adsorbate/slab systems compared to the bare slab, always resulting in high test errors.

In crystallography, the category of a crystal space group give insights into its electronic structure and surface symmetry, which in turn can also affect adsorption energies. Specifically, in FIG. 10E, extremely negative adsorption energies (differential reconstruction) are only observed in the “Ed-3m” space group, while extremely positive energies values (hydrogen desorption) are only associated with the “I-43d” space group. These “I-43d” and “Fd-3m” space group⁰ both have poorly symmetric environments with relatively constrained atomic positions, which can cause hydrogen repulsion resulting in poor catalytic activity or differential reconstruction with catalyst deactivation. Therefore, a well-designed symmetric crystal structure is essential for enhancing the catalytic activity of catalysts, which can promote a uniform distribution of intermolecular forces, ensuring that adsorption or desorption occurs within a moderate range.

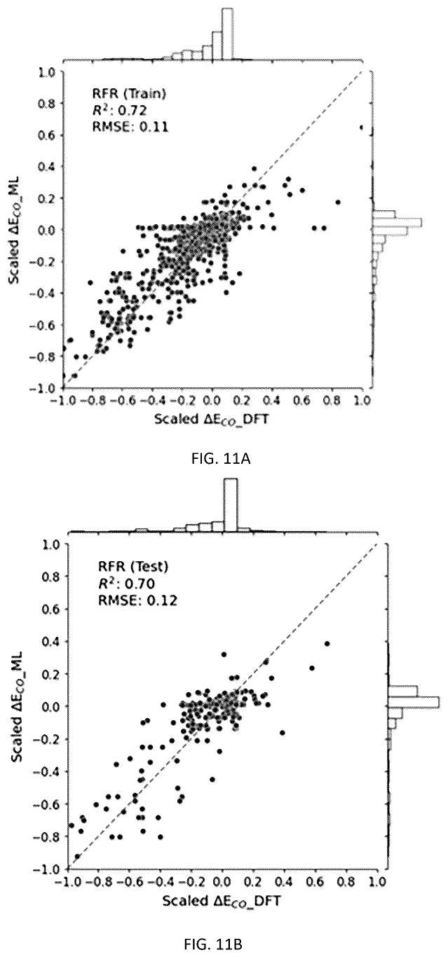

The SISSO approach identifies several key features, labeled as sis1, sis2, sis_co1 (see Eq 4-6 and FIGS. 4C-D). Particularly, the feature of sis_co1 combines three fundamental properties: the distance between CO and its closet neighbor (d_C_1), the average metal-sulfur bond length (avg_mslen) and the energy above the convex hull (Ehull), in a specific formulaic relationship (Eq 6). As shown in FIGS. 11A-B, with only one single feature applied in the ML model, sis_co1 achieves an R² of nearly 70% in predicting CO adsorption energies. This finding suggests that sis_co1 effectively encapsulates and predicts the behavior of chemical adsorption with only one indicator. Through mathematical combination of fundamental physical properties, it is possible to distill complex interactions into a singular, predictive metric, thereby simplifying the model without significantly compromising its accuracy. To understand how these variables in sis_co1 interplay in the CO adsorption energy behavior, FIG. 5D correlates the M-S bond length, d_C_1 and Ehull with the CO adsorption energy. There is a distinct trend where d_C_1 exceeds 1.2 Å, the avg_mslen falls within the ranges of 2.4-3.1 Å, and the Ehull is less than 0.2 eV, a notable accumulation of data points emerges around the −0.67 eV level on the CO adsorption energy, indicating a favorable adsorption state under the given conditions. When the average M-S bond length reaches around 3.2 Å, it leads to very negative CO adsorption energies, similarly to the impact previously observed in FIG. 10B for H adsorption. It indicates that the MₓS_{y} lattices with around 3.2 Å bond length tend to be energetically unfavorable, prompting a structural reorganization for H or CO adsorption. These discoveries reveal the relationship between catalytic activity and fundamental properties and allow for the targeted design of catalysts with optimized structures that enhance activity and maintain stability.

The comparison of ML performances between this work and literature works are presented in Table 4.

TABLE 4
Machine learning studies for adsorption energy prediction on solid surfaces. The machine
learning models developed in this work were compared with other existing studies focusing
on the prediction of adsorption energies. The first row displays the testing error
results for H adsorption and CO adsorption respectively. Features can be mainly categorized
into geometrical (G), electronic (e), and energetic (E) features.

| | Adsorption | | ML | Training | |
|---|---|---|---|---|---|
| Type | system | Feature | model | Data | Error (eV) |
| This | H, CO on MₓS_{y} | SISSI generated; Geo: | RFR | 2683 H/ | R²: 0.77/0.77 |
| work | (100), (110), | atomic distances, angle; | | 1817 CO | RMSE: 0.11/ |
| | (111) facets | Ele: band structure, | | | 0.12 |
| | | electronegativity; | SVM | | R²: 0.69/0.68 |
| | | Ene: Energy above the | | | RMSE: 0.13/ |
| | | convex hull, total energy, | | | 0.13 |
| | | formation energy. | GPR | | R²: 0.70/0.73 |
| | | (FIG. 2 and Table 1) | | | RMSE: 0.13/ |
| | | | | | 0.12 |
| | | | MLP | | R²: 0.69/0.69 |
| | | | | | RMSE: 0.13/ |
| | | | | | 0.12 |
| G | CO on Au (111), | Orbital wise CN | LR | 30 | RMSE: 0.06- |
| | (100), (211) | | | | 0.19 |
| | facets⁷² | | | | |
| | Hon Co₃O₄ | Adjusted CN | LR | 30 | RMSD: |
| | (100), (110), | | | | 0.169 |
| | (111), (211), | | | | |
| | (311) defected | | | | |
| | facets⁷³ | | | | |
| | CO on Ag- | distances + bonds + cluster | RFR | 2,000 | RMSE: 0.17 |
| | alloyed Auₓ(SR)_{y} | graphical + volume | | | R²: 0.78 |
| | nanoclusters¹⁷ | | | | |
| | CO, H on pure | Atomic graph + atomic | CNN | 12,000 | MAE: 0.13- |
| | metals, metal | properties + Voronoi | | | 0.19 |
| | alloys, | polyhedra-neighbor | | | |
| | intermetallic | | | | |
| | surfaces⁷⁴ | | | | |
| | CO, H on metal | Crystal graph + atomic | CNN | 14,769 H | MAE: 0.08- |
| | alloys⁷⁵ | properties + labeled site | | 20,811 CO | 0.13 |
| | CO, HOCO on | Atomic positions + | ANN | 1,400 | RMSE: 0.05- |
| | Au surfaces⁷⁶ | symmetry functions | | | 0.06 |
| | CO, H on metal | Atomic number, | Active | 1,684,908 | MAE: 0.17- |
| | alloy⁵³ | electronegativity, CN, E_{ad} | ML | | 0.46 |
| | | on the pure metal | | | |
| e | CO on | atomic number, radius, | XGBR | 171 | R²: 079-0.90 |
| | metal-nonmetal | electronegativity, d- | | | RMSE: 0.23- |
| | codoped | electron, ionization energy | | | 0.16 |
| | graphene⁷⁶ | | | | |
| | CO on (100) | d-band characteristics + | ANN | 250 | RMSE: 0.12 |
| | bimetallic | electronegativity | | | |
| | alloys⁷⁷ | | | | |
| | CO on bimetallic | LMTO d-band center + | KRR | 260 | RMSE: 0.08 |
| | alloys⁷⁹ | electronegativity | | | |
| | C, O, H | Atomic, bulk properties + | CS | 900 | RMSE: 0.15 |
| | containing | CN + work function + d- | | | |
| | adsorbates on | band, sp-band | | | |
| | single atom and | | | | |
| | bimetallic | | | | |
| | alloys⁷⁹ | | | | |
| | H, C, N, O, S, | Density of States | CNN | 37,000 | MAE: 0.116 |
| | and | | | | |
| | hydrogenated | | | | |
| | counterparts on | | | | |
| | bimetallic alloy | | | | |
| | surfaces⁴⁰ | | | | |
| E | H on TM doped | Vacancy formation energy | LR | 40 | MAE: 0.16 |
| | (111) (311) | | | | |
| | Co₃O₄ facets⁸⁰ | | | | |
| | 71 molecular | Ead of O, OH, CCHOH | PCR | 31,000 | MAE: 0.12- |
| | fragments from | | | | 0.19 |
| | C1 and C2 | | | | |
| | alcohols on | | | | |
| | transition | | | | |
| | metals⁸¹ | | | | |
| | H, C, N, O, S, | Ead of various species | GPR | 37,000 | RMSE: 0.09- |
| | CH, CH₂, CH₃, | | | | 0.27 |
| | NH, OH, SH on | | | | |
| | bimetallic alloy⁸² | | | | |

CN: coordination number; LR: Linear regression; KRR: Kernel ridge regression; CNN: Convolutional Neural Network; CS: Compressed sensing; PCR: Principal Component Regression; XGBR: extreme gradient boosting regression; MAE: mean absolute error, closer to 0 indicates better performance; RMSE: root mean squared error, closer to 0 indicates better performance; R²: the coefficient of determination, closer to 1 indicates better performance.

It is obvious from Table 2 above that ML models employed for predicting adsorption energies mostly struggle to achieve high accuracy. This difficulty can be primarily attributed to three pivotal factors in the field of materials science. First, the limited availability of large training datasets still poses a great challenge for the model to learn the intricate relationships between basic features and chemical adsorption behaviors. Second, the differential reconstruction during DFT optimization poses a large error during the ML prediction as mentioned above. And third and more crucially, in DFT, even minor variations in the initial structures can lead to significantly different energy output values; this is due to the differences in the distribution of electron clouds, or the systems involving multiple minima states with complex energy landscapes, where DFT calculations may struggle to find the global minimum energy state. For example, the largest discrepancy in predicted H adsorption energies was observed on the Zr₅S₈ (110) facet (as shown in FIG. 6E-F). The DFT calculated result for the structure in FIG. 6E was −2.69 eV, and the structure in FIG. 6F was very similar to FIG. 6E, yielding an ML-predicted energy of −2.08 eV but the actual calculated DFT result was significantly lower, at −7.03 eV. Such instances underscore the complexity of accurately modeling adsorption energies due to the sensitive dependence of DFT on the precise atomic configuration and the electronic structure of the material. As it is common for different local minima to exhibit variations in adsorption energy exceeding 1 eV, these slight differences in the arrangement of atoms or electron density introduce a large noise into any ML model that does not account for it.

### Large Scale Screening for Metal Sulfide Catalysts

To select the most suitable materials for electro/photo-catalytic HER and CO₂RR, the screening procedure schematized in FIG. 1 was followed. The well-trained RFR models were implemented to classify the stable structures and predict the H/CO adsorption energy for the remaining MₓS_{y} materials that had not been previously calculated using DFT. Based on these screening steps, the pool of potential candidates was quickly narrowed down, leading to the development of more efficient and sustainable catalysts for HER and CO₂RR.

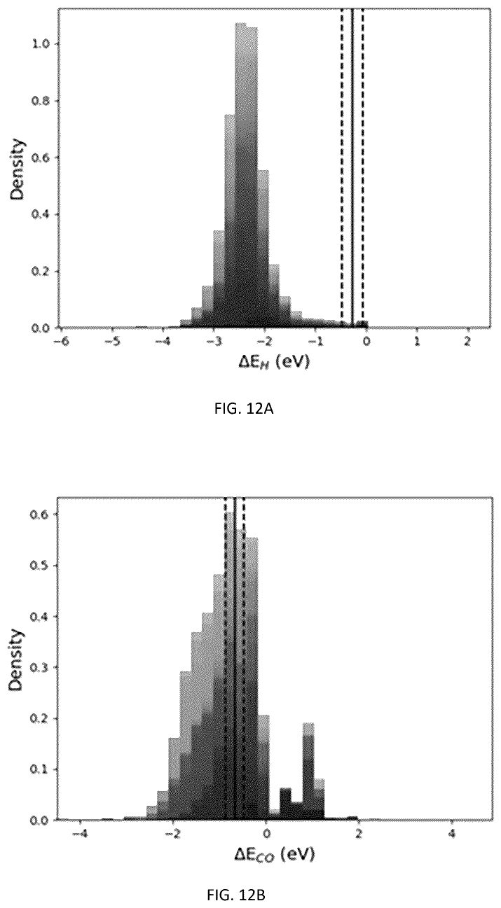

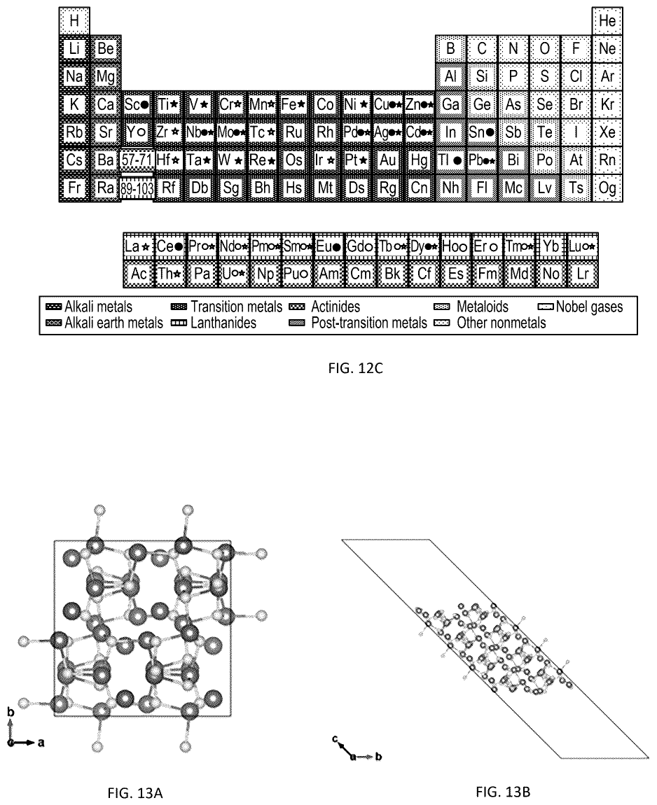

The distributions of adsorption energies (predicted by ML and calculated by DFT) is presented in FIGS. 12A-B. There are 38 different MₓS_{y} lattices within the ideal H adsorption energy range for HER, while 77 MₓS_{y} lattices fall within the ideal CO adsorption energy range for CO₂RR. The screening results of different adsorption sites and surfaces in MₓS_{y} lattice systems are shared in the figures. Additionally, the screened materials for HER and CO₂RR were marked within the periodic table in FIG. 12C. The histograms in FIGS. 12A-B reveal that transition metals from the 5th and 6th periods (highlighted in purple and red) have significant contributions, which are known for their variable oxidation states and ability to form complex compounds. The H adsorption energies are mostly centered around −2.5 eV, forming a near-normal distribution with a slight tail, indicating uniform adsorption strength across all these materials. But the distribution of CO adsorption energies in FIG. 12B shows a stepped pattern, suggesting that adsorption strengths are clustered at specific energy levels and materials with similar electronic configurations would exhibit similar bonding environments and adsorption behaviors. Metals from earlier periods (darker colors) tend to have higher densities near 0 eV, generally exhibiting weaker CO adsorption strengths.

Additionally, alkali and alkaline earth metal sulfides are not recommended as they are soluble in water or easily hydrolyzed upon contact with water⁰. The suitable MₓS_{y} predominantly include transition metals, which are commonly used in catalysis due to their ability to adopt multiple oxidation states and facilitate electron transfer. Transition metals and Lanthanides have partially filled d-orbitals or f-orbitals that provide a rich electronic structure to participate in bonding with adsorbates and stabilize the intermediate species of H or CO. Notably, the local electronegativity (e_H/C_loc) values of desirable materials are mostly around 2, indicating a preference for having both metal and sulfur atoms surrounding the H/CO atom. In other words, a metal sulfide surface with only one atom type on the first layer may exhibit lower stability and less desirable catalytic performance. Because the bond length between adsorbate and substrate is strongly influenced by the substrate's sp-electron density⁰, a more uniform sp-electron density on the first layer is potentially less favorable to interact with intermediates. Furthermore, the presence of the (111) facet is very rare among the high-performance materials, it typically has a more closely packed arrangement and less stability compared to (100) and (110) facets.

In the present disclosure, the screened materials are clearly annotated regarding whether they have been experimentally synthesized and studied for these HER or CO₂RR applications up to date. A summary of the novel materials and the most common catalysts are also provided below in Table 5, similar to Tables 1 and 2.

There are limited options for HER due to the low catalytic activity and high cost of many materials. Analysis indicates that both lanthanides and actinides are prohibitively expensive⁰ (see FIG. 12C), making CdS, PbS, ZnS, MOS₂ and SnS₂ viable and cost-effective choices⁰ for both photo- and electrocatalytic HER⁰. Among the listed sulfide materials, CdS and ZnS are the most affordable coupled with their desirable chemical properties⁰, which also aligns with experimental findings as described herein. Additionally, CuS, CeS, SinS, YS, and NbS₂ are predicted to be potential efficient and affordable electrocatalysts with zero band gap, among these, SinS and YS have not yet been experimentally tested for electrocatalytic HER. The relatively costly materials such as GdS₂, US₃, NdS, PrS₂, ErS, TbS, HoS, Tm₅S₇, LuS, PmS₂, and PuS₂, have also been proposed as novel materials for their utilization in HER as referred in the SI file.

A broader range of materials are proposed for both photo- and electrocatalytic CO₂RR, including FeS₂, CdS, PbS₂, ZnS, TiS₂, LaS₂, NiS₂, SmS₂, MOS₂, VS₄, ZrS₂, and WS₂. In addition, MnS₂, CUS, Cu₂S, CrS₂, and NbS₂ are pinpointed as suitable only for electrocatalysis. Notably, the cost-effective materials such as MnS₂, LaS₂, CrₓS_{y}, SmS₂, and ZrₓS_{y} have not yet been investigated in this application, presenting their untapped potential. According to the references listed in the SI file, CuS is the most studied catalyst, followed by MoS, WS₂, NiS₂, CdS, ZnS, FeS₂, PbS₂, TiS₂, and Ag₂S, which is consistent with our prediction that there are more active sites on these surfaces. As summarized in several review papers of CO₂RR^{0,0}, CuₓS_{y} electrocatalysts tends to have higher selectivity towards narrower product varieties, especially for formic acid production, CdS shows 100% reaction selectivity for CO, MoS₂ and TiS₂ also demonstrate high selectivity for CO production with Faradaic efficiency of 83% and 100%, respectively. Therefore, the consistency between ML predictions and experiments validates the predictive power and lends credibility to the ML models. For future work, the unstudied materials screened from this work are promising candidates for research and experimentation in the field of photo- or electro-catalytic HER and CO₂RR, exploring their properties and advancing the development of high-performance catalytic frameworks.

Overall, a screening procedure was conducted for searching optimal metal sulfide catalysts for HER and CO₂RR by combining DFT with ML techniques. DFT was introduced to obtain the convergence capacity of 4110 adsorption structures and the H and CO adsorption energies on the surfaces, serving as targets for supervised ML models. RF outperformed other ML algorithms with a higher accuracy (0.92) for stability classification and R² (0.77) for adsorption energy regression task. Therefore, these well-trained RF models allow us to perform a fast large-scale material screening to identify potential candidates among 11076 unique H and CO adsorption structures on metal sulfide facets (6966 predicted by ML, 4110 calculated from DFT). Finally, we identified 38 and 77 potential materials with optimal stability, light-utilization efficiency and catalytic activity for HER and CO₂RR, respectively.

By interpreting the ML model, a predictive relationship was established between the fundamental physical features and intermediate adsorption energies. The local environment of the adsorbate significantly affects the strength of adsorption. Even a single feature generated by SISSO can roughly predict the CO chemisorption behavior. Furthermore, three key reasons were identified to explain why current ML models struggle to achieve a perfect replication of DFT results. Therefore, using ML with molecule modeling techniques can provide plausible explanations and clear insights of physical phenomenon, enables to guide the design for optimal catalyst for HER and CO₂RR.

### Computational Details

The screening process involved three steps, namely stability, band gap, and intermediate adsorption energy analyses. First, phase stability plays a critical role in determining the material's synthesizability and it was assessed using convex hull analysis with the energy above the convex hull (Ehull). It can determine the material's synthesizability, phase stability and its potential for degradation under certain operating conditions⁰. Positive Ehull values indicate decreasing stability, while an Ehull of zero signifies thermodynamically stable compounds.

Additionally, the convergence of DFT structural optimization from RF classification is also set as a criterion during this step. These two criteria (both Ehull and DFT convergence) help ensuring reasonable initial guess for H and CO adsorption structures. Therefore, we set Ehull less than 0.01 eV and ML classified to be truly converged as criteria for the first screening step. Secondly, the lattices were filtered based on their band gap values. The band gap energy of a catalyst determines the energy required to excite electrons from the valence band to the conduction band, which initiates electron transfer and drives the photocatalytic reactions^{0,0}. A smaller band gap also indicates higher electron-transfer dynamics⁰, making it more favorable for electron transfer and promoting electrochemical reactions. Since DFT calculations are known to have underestimations in representing band gaps, we set the range to 0˜3 eV (in absolute value) to ensure that the selected materials have a high likelihood of capturing solar light as photocatalysts or achieving good charge transfer capacity as electrocatalysts⁰. Furthermore, to predict the activity and selectivity of metal sulfide catalysts for HER and CO₂RR, a relationship between descriptors and activity was established based on the free energy change of intermediate adsorption (ΔG_{H} and ΔG_{CO}). The optimal adsorption energy indicates an ideal balance between reactant adsorption resistance and the ease of proton detachment or products desorption from the catalyst surface^{0,0}. The relationship revealed that an optimal activity and selectivity can be achieved with a ΔG_{H} of −0.03 eV, which corresponds to a ΔE_{H} of −0.27 eV⁰. For the screening of catalysts for CO₂RR, relations were used to predict the optimal performance with ΔG_{CO} of −0.17 eV, which corresponds to a target ΔE_{CO} of −0.67 eV^{0.0}. The physical understanding behind these values is that for HER, elements that can easily adsorb and desorb H are crucial, while for CO₂RR, elements that can stabilize the intermediate species of CO₂ reduction are important.

First principle calculations presented in this work were carried out using the plane wave-based DFT method as implemented in the Vienna ab Initio Simulation Package (VASP)⁰. DFT calculations with a dispersion correction method (DFT-D3) were performed to account for van der Waals interactions of adsorption and dissociation on the different surfaces. Long-range dispersion forces were obtained using Grimme's method⁰. The generalized gradient approximation with the Perdew-Burke-Ernzerhof functional (GGA-PBE) was used to calculate the energy⁰. Electron-ion interactions were described using the projector augmented wave (PAW) method⁰. An energy cutoff of 500 eV for the plane-wave basis set was used for the convergence of the total energy. The convergence criteria were set to 10⁻⁵ eV and 0.02 eV/Å for the electronic self-consistent iteration and the forces on each atom, respectively, while the Brillouin zone was sampled using Monkhorst-Pack mesh k-points with a reciprocal space resolution of 2π×0.04/Å.

For the last screening process, 887 crystal structures were collected from Materials Project (MP) database⁰. The pymatgen package⁰ was then utilized to create three general facets (i.e., 100, 110, and 111) for these lattices and generate a diverse set of H and CO adsorption structures for all possible adsorption sites. Noted that the surfaces predicted by pymatgen with more than 50 sites are always invariably complex and completely precludes convergence within DFT simulations, which were subsequently removed to save time and computational resources. The structure of In₅S₄ lattice in FIGS. 13A-B is presented as an example, the extensive number of sites in In₅S₄ lattice with intricate electronic and atomic interactions would complicate the optimization landscape.

All ML algorithms were conducted by the open-source code Scikit-learn⁰ package in the Python (version 3.10.5) environment. The calculated adsorption energies were randomly split as training and testing sets according to an 80:20 ratio. Random search, where hyperparameters are randomly sampled from a defined range or distribution, was employed for using cross-validation to estimate the performance of each hyperparameter combination. Four different supervised ML algorithms were selected in this work, including: MLP⁰ (a backpropagation algorithm), RFR⁰ (highly effective with categorical data by aggregating predictions from numerous trees), SVM⁰ (a versatile algorithm by finding an optimal hyperplane), and GPR⁰ (a non-parametric, kernel-based Bayesian approach that models the relationship between input and output variables as a probability distribution). All the optimized parameters for ML models are summarized in Table 6.

TABLE 6
The hyper-parameters of ANN, RF, SVM, and GP algorithms for
classification (C) and regression (R) tasks were fine-tuned
by randomized search with 5-fold Cross-Validation.

| Models | Types | | Parameters |
|---|---|---|---|
| MLP | C | H/CO | solver = ‘adam’, hidden_layer_sizes = (150), max_iter = 1000, |
| | | | activation = ‘relu’ |
| | R | H | solver = ‘sgd’, hidden_layer_sizes = (96, 61, 25), activation = ‘relu’, |
| | | | alpha = 0.4, tol = 1e−6, learning_rate_init = 0.05, max_iter = 10000, |
| | | | learning_rate = ‘adaptive’ |
| | | CO | solver = ‘adam’, hidden_layer_sizes = (41, 74), activation = ‘relu’, |
| | | | alpha = 0.07, tol = 1e−6, learning_rate_init = 0.01, max_iter = 10000, |
| | | | learning_rate = ‘constant’ |
| RF | C | H/CO | n_estimators = 250, max_depth = 50, random_state = 42 |
| | R | H | n_estimators = 257, max_depth = 30, min_samples_split = 2, |
| | | | random_state = 120 |
| | | CO | n_estimators = 282, max_depth = 40, min_samples_split = 6, |
| | | | random_state = 120 |
| SVM | C | H/CO | kernel = ‘rbf’, C = 1, gamma = 0.01, random_state = 42 |
| | R | H | kernel = ‘rbf’, C = 1.6, gamma = 0.04, epsilon = 0.01, degree = 2, |
| | | CO | kernel = ‘rbf’, C = 1.6, gamma = 0.04, epsilon = 0.1, degree = 2, |
| GP | C | H/CO | n_restarts_optimizer = 1, kernel = RBF |
| | R | H/CO | n_restarts_optimizer = 10, alpha = 0.08, kernel = 1**2 * |
| | | | RationalQuadratic(alpha = 0.1, length_scale = 1) |

Data preprocessing included the preparation and transformation of raw data for ML model training, handling missing values, removing outliers, potentially extracting or selecting features, and encoding categorical variables, to ensure it is optimized for the effective training of ML models. Initially, data points with adsorption energy values outside the range of −11 to 6 eV were disregarded, as these are deemed outliers that could degrade the quality and reliability of the model's predictions. Adsorption energies that fall outside this range are mainly due to the reconstruction of structures after structural optimization or hydrogen desorption from the surface, where the surface geometry changes significantly. RF importance ranking analysis with mean decrease impurity (MDI) was conducted to handle both continuous and categorical features. Additionally, features with higher MDI scores in the RF model are considered as more important and relevant to ML prediction.

In order to evaluate the prediction performance of each model, mean-square error (RMSE), and coefficient of determination (R²) for regression, have been calculated for the training and testing sets. The formula for each one of the indicators is as follows:

$$RMSE = \sqrt{\frac{1}{m} {\sum }_{i = 1}^{m} {( {y}_{ti} - {y}_{pi} )}^{2}} \qquad ( 1 )$$

$${R}^{2} = 1 - \frac{{\sum }_{i = 1}^{m} {( {y}_{ti} - {y}_{pi} )}^{2}}{{\sum }_{i = 1}^{m} {( {y}_{ti} - {\overline{y}}_{ti} )}^{2}} \qquad ( 2 )$$

where yₜᵢ is the adsorption energy value from DFT calculations randomly selected from the test set, yₚᵢ is the predicted value of the corresponding regression model, y̅ₜᵢ is the average value of yₜᵢ, m is the number of samples in the dataset. Generally, an ideal model should have R² value close to 1, and a small RMSE close to 0.

To provide a more efficient description of the local charge environment and quantify the driving force of electron transfer at an adsorption site, we proposed the use of the local electronegativity e_H/C_loc as:

$$e\_H / C\_loc = {\prod }_{j = 1}^{N} {{e}_{j}}^{\frac{1}{N}} \qquad ( 3 )$$

where eⱼ is the Pauling electronegativity of atom j in the neighbor list on adsorbates H/CO, and N is the total number of atoms within the defined neighboring shell including the adsorption site.

SISSO operates by initially screening the features using a measure of independence, such as mutual information, to identify the most meaningful ones, and subsequently applying a sparsifying operator that enforces sparsity and further reduces the feature space. Based on this technique, three top-ranked descriptors (i.e., sis1, sis2, sis_co1) were calculated as:

$$sis 1 = Ehull \times d\_xy \_ 1 / exp ( d\_xy \_ 1 ) \qquad ( 4 )$$

$$sis 2 = {Ehull}^{3} / \vert d\_xy \_ 2 - n\_atoms \_top \vert \qquad ( 5 )$$

$$sis\_co1 = ( d\_C \_ 1 \times avg\_mslen ) / ( Ehull - avg\_mslen ) \qquad ( 6 )$$

where Ehull is the energy above the convex hull of bulk lattice, d_xy_1 and d_xy_2 are the distance between H/CO and its 1^{st} and 2^{nd} neighbors from xy direction, n atoms top is the number of atoms in the top layer of the surface and avg_mslen is the average metal-sulfur (M-S) bond length.

### Example 2

Theoretical screening using a combined Machine Learning and Density Functional Theory framework identified transition-metal sulfides with optimal adsorption energies for hydrogen and carbon monoxide, ensuring balanced surface binding and enhanced catalytic stability. For electrochemical CO₂ reduction to CO, 24 candidate materials were initially proposed from computational modeling. Among them, MnS₂, V₃S₄, and Ni₃S₂ were selected for experimental validation to demonstrate the accuracy and predictive strength of the theoretical model, owing to their favorable electronic structures, moderate bandgaps, thermodynamic stability, ease in synthesis and economic value. These three catalysts are explored for this application for the first time in this study, guided by the modeling prioritization, and compared to CdS, CuS, and MoS₂ as literature benchmarks. Electrochemical measurements confirmed that MnS₂ and V₃S₄ exhibited distinct CO₂-reduction activity with Faradaic efficiencies of ˜45% and ˜41% and selectivity toward CO exceeding exceeded 90%. The other electrocatalysts (CdS, CuS, and MoS₂) were dominated by the hydrogen evolution reaction (HER) as a side reaction and relatively inactive for CO₂ conversion.

In photocatalytic tests of CO₂ conversion, 20 candidates initial catalysts were proposed and then MnS₂ and VS₄ were selected for experimental validation. It shows that VS₄ outperformed MnS₂ under high-pressure CO₂, showing higher CO yield and stability.

Overall, these results experimentally validate the theoretical predictions and demonstrate that ML-guided catalyst discovery effectively identifies materials with superior selectivity and performance for CO₂-to-CO conversion in electrochemical and photocatalytic conversions.

### Experimental Results: Electrochemical CO₂RR to CO

Cyclic voltammetry (CV) measurements were performed in a standard three-electrode configuration using a graphite sheet modified with the respective metal sulfide catalysts. An Ag/AgCl electrode (saturated KCl) and a Pt mesh served as reference and counter electrodes, respectively. The electrolyte was 0.25 M NaHCO₃ prepared with high-purity Milli-Q water and pre-saturated by purging either Ar (blank) or CO₂ gas for 30 minutes prior to each experiment. During the CV scans, continuous bubbling of the respective gas was maintained to ensure a saturated environment and prevent mass transport limitations. All voltammograms were recorded at a scan rate of 20 mV·s⁻¹ in the potential window of 0 to −1.5 V_{Ag/AgCl}. To normalize for intrinsic activity, current densities were corrected by the electrochemically active surface area (ECSA) obtained from capacitive current measurements in a non-faradaic region.

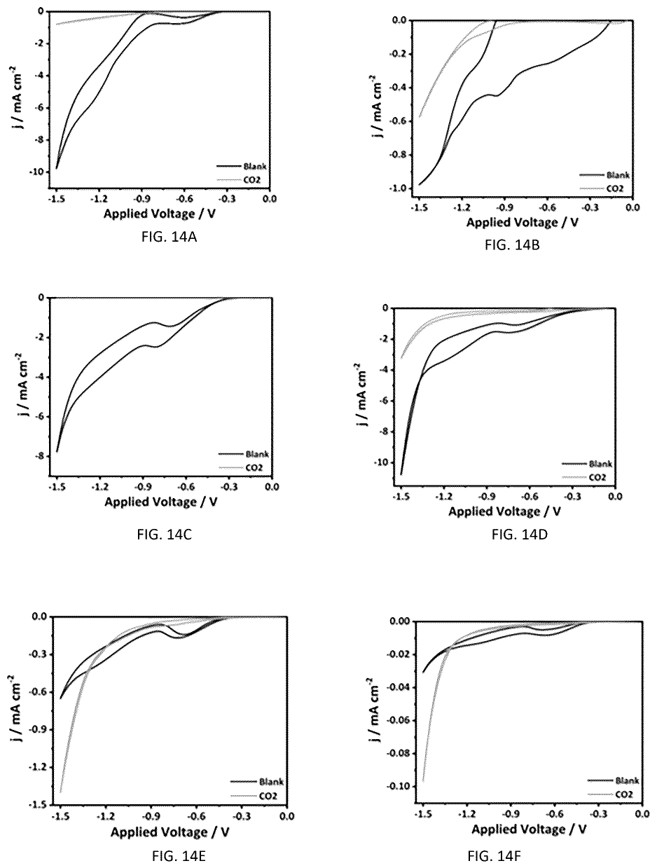

Cyclic voltammetry in 0.25 M NaHCO₃ with continuous bubbling of either Ar (blank) or CO₂ and with all currents normalized by ECSA reveals two distinct behaviors across the six transition-metal sulfides (FIGS. 14A-F). CV curves of transition-metal sulfides in CO₂- and Ar-saturated 0.25 M NaHCO₃ at 20 mV·s⁻¹ are shown in FIGS. 14A-F. CV curves are shown for (FIG. 14A) CdS, (FIG. 14B) CuS, (FIG. 14C) MoS₂, (FIG. 14D) Ni₃S₂, (FIG. 14E) MnS₂ and (FIG. 14F) V₃S₄. Currents densities reported are based on ECSA. Under CO₂ saturation (red curves), CdS, CuS, MoS₂, and Ni₃S₂ exhibit suppressed cathodic currents compared with Ar-saturated (black curves) and lack a CO₂-specific knee or transport plateau, indicating HER-dominated behavior and surface deactivation under CO₂. In contrast, V₃S₄ and MnS₂ retain or enhance cathodic response, consistent with distinct CO₂-reduction activity. CdS, CuS, MoS₂, and Ni₃S₂ show pronounced suppression of cathodic current in the CO₂-saturated electrolyte (j_{CO}_{₂}) over the entire window (FIGS. 14 A-D). At −1.4 V_{Ag/AgCl}, the differences j_{CO}_{₂}−j_{Ar}(=Δj) are +6.77, +0.49, +5.54, and +3.62 mA cm⁻², respectively, i.e., the Ar blanks (j_{Ar}) are substantially more cathodic (Table 7).

TABLE 7
Summary of cathodic current densities* for transition-metal sulfides
in Ar- and CO₂-saturated 0.25M NaHCO₃ at −1.4V _{Ag/AgCl}.

| | Ar-saturated Blank | CO₂-saturated | | |
|---|---|---|---|---|
| | j_{Ar} | j_{CO2} | Δj | In |
| Electrocatalyst | mA/cm² | mA/cm² | mA/cm² | Disclosure |
| CdS | −7.43 | −6.57 × 10⁻¹ | 6.77 | No |
| CuS | −8.85 × 10⁻¹ | −3.94 × 10⁻¹ | 4.91 × 10⁻¹ | No |
| MnS₂ | −4.92 × 10⁻¹ | −7.21 × 10⁻¹ | −2.29 × 10⁻¹ | Yes |
| MoS₂ | −5.55 | −1.18 × 10⁻² | 5.54 | No |
| Ni₃S₂ | −5.43 | −1.81 | 3.62 | Yes |
| V₃S₄ | −2.07 × 10⁻² | −3.43 × 10⁻² | −1.36 × 10⁻² | Yes |

*Measured current densities (j_{Ar} and j_{CO2}) are normalized by electrochemically active surface area (ECSA). The difference (Δj = j_{CO2} − j_{Ar}) indicates the net influence of CO₂ saturation on cathodic activity. Positive Δj values denote current suppression under CO₂ (HER-dominated behavior), while negative Δj values correspond to enhanced current (indicative of CO₂-reduction activity).

The Ar CV curves for these electrocatalyst exhibit large HER-type waves beginning near −1.0 V_{Ag/AgCl} with broad shoulders consistent with hydrogen evolution and, for Cu- and Ni-sulfides, concurrent surface reconstruction. Under CO₂ the curves collapse toward capacitive baselines and introduce no additional cathodic feature, indicating that CO₂/HCO₃⁻ deactivates these surfaces rather than opening a new faradaic pathway. The matched hydrodynamics rule out a mass-transfer artifact. The fact that CO₂ lowers the bulk pH by ˜1.5 units (thus making the CO₂ scans ˜90 mV more negative on the RHE scale at the same external potential) underscores that the loss of current is chemical-consistent with carbonate/CO₂ adsorption that inhibits the Volmer step, rapid formation of resistive oxy-sulfide/hydroxide films, and CO blocking of transiently formed metal-rich sites rather than a simple reference-scale effect. No CO₂-specific knee or transport plateau is observed for CdS, CuS, MoS₂, or Ni₃S₂. Therefore, based on their CV curves, it is reasonable to infer that these materials behave as HER-dominated electrodes in bicarbonate electrolyte.

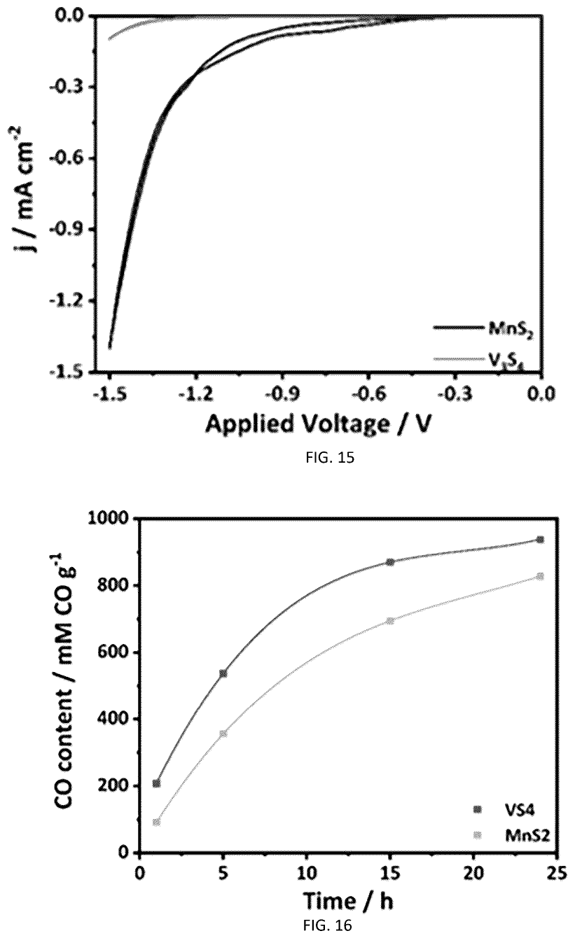

By contrast, MnS₂ and V₃S₄ display higher cathodic currents when CO₂ is present under otherwise identical conditions (FIGS. 14E-F). For MnS₂ the difference at −1.4 V is j_{CO}_{₂}-j_{Ar}=−0.229 mA cm⁻², and for V₃S₄ the CO₂ trace is reproducibly ˜1.5× larger than the Ar control at the same potential (Table 4). Because both gases were bubbled continuously, the divergence cannot be ascribed to hydrodynamics, and because the other four sulfides are strongly suppressed in CO₂, the modest increase seen only for MnS₂ and V₃S₄ is not a universal pH/referencing artifact. The additional current appears only at more negative potentials and grows toward −1.4 V_{Ag/AgCl} without the broad HER shoulders that characterize the Ar blanks, consistent with a CO₂-dependent faradaic channel. We therefore attribute the CO₂-specific enhancement on MnS₂ and V₃S₄ to the onset of CO₂ reduction to CO, while HER remains the principal process in the Ar electrolyte. The absolute magnitudes are modest on the CV curves, but after ECSA normalization and with matched gas flow they constitute the only clear electrochemical signatures of CO₂ activation within these materials set. The CO₂-saturated CVs of MnS₂ and V₃S₄(FIGS. 14E-F) reveal pronounced cathodic activity attributed to CO₂ reduction, with MnS₂ exhibiting a slightly earlier onset and higher current density than V₃S₄, indicating its superior intrinsic activity under identical conditions (FIG. 15). FIG. 15 provides a comparison of CO₂-saturated CV for the CO₂-active sulfide catalysts V₃S₄ and MnS₂ in CO₂-saturated 0.25 M NaHCO₃ at a scan rate of 20 mV·s⁻¹.

Taken together, the comparative CVs separate the sulfides into two groups. Ni₃S₂, MoS₂, CdS, and CuS are inhibited by CO₂/HCO₃⁻ and show no evidence of a CO₂-reduction wave, identifying them as HER-dominated under these conditions. In contrast, MnS₂ and V₃S₄ exhibit CO₂-specific increases in intrinsic cathodic current that can be ascribed to CO₂ reduction possibly to CO. These trends, obtained under ECSA normalization and matched bubbling, point to MnS₂ and V₃S₄ as the most promising CO-selective candidates for further steady-state and product-analysis studies.

Chronoamperometric (CA) studies were carried out for 24 hours at −1.4 V_{Ag/AgCl} using MnS₂ and V₃S₄ electrocatalyst in 0.25 M NaHCO₃ under both CO₂- and Ar-saturated conditions. Following electrolysis, the gases were collected and analyzed using gas chromatography equipped with thermal conductivity and flame ionization detectors (GC-TCD/FID). The CO production rates for MnS₂ and V₃S₄ were determined to be 4.6±1.2 mol⁻¹ cm⁻² and 0.26±0.51 mol⁻¹ cm⁻², respectively. The corresponding Faradaic efficiencies for CO formation were 45 9% for MnS₂ and 41.2±7% for V₃S₄. Under the applied potential, both catalysts maintained >90% selectivity toward CO, indicating stable and preferential CO₂ reduction over competing hydrogen evolution.

### Experimental Results: Photocatalytic CO₂-to-CO

Photocatalytic CO₂-to-CO experiments were performed in a stainless-steel high-pressure batch photoreactor equipped with an optical window (sapphire) and gas sampling ports. Prior to each run the vessel was leak-tested and purged three times with CO₂. The reactor was charged with 50 mg of catalyst (either VS₄ or MnS₂) and magnetically stirred throughout the experiment at ambient temperature. CO₂ from a commercial cylinder was introduced until the gauge read ˜56-58 bar at room temperature. At these conditions CO₂ is in the liquid phase and typical cylinder pressures are 45-65 bar at ambient temperature, consistent with the CO₂ saturation pressure near 22-25° C. Illumination was supplied continuously through the window by a solar simulator with 1000W power. Each experiment ran for 24 h, and gas samples were withdrawn periodically in a gas bag via a gas-tight sampling loop. Products were quantified on a GC fitted with TCD and FID. CO was quantified against external standards. CO yields were normalized to catalyst mass and reactor volume.

FIG. 16 illustrates photocatalytic CO₂-to-CO conversion over VS₄ and MnS₂ under high-pressure CO₂ Conditions: 56-58 bar CO2, 1000 W solar simulator, 24 h, 50 mg of catalyst. The time-dependent CO accumulation profiles in FIG. 16 show that VS₄ outperforms MnS₂ across the entire 24 h window. The VS₄ CO content curve rises more steeply at early times and maintains a higher production rate before approaching a slower, near-asymptotic region, whereas MnS₂ exhibits a lower initial slope and a lower final cumulative yield. The deceleration observed for both materials at longer times is consistent with mass-transfer limitations in a dense medium, depletion of accessible interfacial CO₂ at the catalyst surface, and/or partial surface passivation by carbonate/bicarbonate or sulfur-oxide species-effects. The higher activity of VS₄ is reasonable given the favorable light absorption and charge-transport characteristics of vanadium sulfides and their propensity to host S-vacancy/edge states that adsorb and convert CO₂. By contrast, MnSₓ materials can suffer from faster electron-hole recombination unless defect chemistry or heterojunctions are engineered.

Placing these results in context, operating under elevated CO₂ pressure is a recognized strategy to increase CO₂ availability at the catalyst-liquid interface and to intensify rates. Pressurized photoreactors operated up to ˜20 bar in water have been shown to boost the formation of CO, CH₄, and liquid products by improving CO₂ solubility and mass transfer, relative to ambient-pressure systems⁸³⁻⁸⁵. Single-phase supercritical CO₂ media can further alter interfacial transport and selectivity. For example, homogeneous and heterogeneous systems have achieved efficient CO formation in scCO₂ or scCO₂-cosolvent mixtures, although such conditions require T>31° C. and p>73.8 bar-distinct from the ambient-temperature, liquid-CO₂ conditions used here⁸⁶⁻⁸⁸.

Overall, the figure demonstrates a clear performance advantage for VS₄ over MnS₂ for photocatalytic CO formation under liquid-CO₂ conditions at ambient temperature. Together with literature showing that pressurization enhances CO₂ availability and that defect-rich sulfides favor the CO pathway, these data position VS₄ as the more promising sulfide platform for further optimization, e.g., through vacancy control, co-catalyst deposition, or heterojunction construction.

### EMBODIMENTS

The present disclosure may also be understood by the following embodiments.

Embodiment 1: A method of converting CO₂ to CO, the method comprising: photocatalytically converting CO₂ to CO in the presence of a catalyst, wherein the catalyst comprises a metal sulfide catalyst, wherein the selectivity toward CO is 90% or greater, and wherein distinct CO2-reduction activity has a Faradaic efficiency of at least 35%.

Embodiment 2: The method of Embodiment 1, wherein the metal sulfide catalyst comprises Ni₃S₂, MnS₂, VS₄, V₃S₄, or combinations thereof.

Embodiment 3: The method of Embodiment 1, wherein the metal sulfide catalyst comprises MnS₂.

Embodiment 4: The method of Embodiment 1, wherein the metal sulfide catalyst comprises V₃S₄.

Embodiment 5: The method of Embodiments 1-4, wherein the method is conducted under a CO₂ pressure from 40 to 70 bar.

Embodiment 6: The method of Embodiments 1-5, wherein the method is conducted for a period of time from 30 minutes to 48 hours.

Embodiment 7: The method of Embodiments 1-6, wherein the method is conducted with 5 to 100 mg catalyst.

Embodiment 8: The method of Embodiments 1-7, wherein the metal sulfide catalyst has a cathodic current density in an Ar-saturated blank (j_{Ar}) from −6.000 to −0.020 mA/cm².

Embodiment 9: The method of Embodiments 1-8, wherein the metal sulfide catalyst has a cathodic current density in a CO₂-saturated blank (j_{CO2}) from −2.000 to −0.030 mA/cm².

Embodiment 10: The method of Embodiments 1-9, wherein a difference (Δj) between a CO₂-saturated blank (j_{CO2}) a cathodic current density in an Ar-saturated blank (j_{Ar}) is from −0.020 to 4.000.

Embodiment 11: The method of Embodiments 1-10, wherein the method is conducted using a graphite sheet modified with the metal sulfide catalyst.

Embodiment 12: The method of Embodiments 1-11, wherein the method is conducted using an Ag/AgCl electrode.

Embodiment 13: A method for predicting CO chemisorption on a surface using metal sulfide catalysts, the method comprising: a) inputting CO-surface distances, metal-sulfur bond lengths and energies above hull; b) predicting the CO chemisorption on a surface; c) setting a minimum acceptable chemisorption on a surface; and d) testing metal sulfide catalysts that meet the predicted minimum acceptable chemisorption.

Embodiment 14: The method of Embodiment 13, wherein the predicting is conducted by applying machine learning algorithms.

Embodiment 15: The method of Embodiment 14, wherein at least two machine learning algorithms are used.

Embodiment 16: The method of Embodiment 14, wherein four machine learning algorithms are used.

Embodiment 17: The method of Embodiment 14, wherein Random Forest, trained with data from Density Functional Theory is used.

Embodiment 18: The method of Embodiments 13-17, wherein two physical inputs are also input.

Embodiment 19: The method of Embodiment 18, wherein the two physical inputs are: energy above the convex hull of bulk lattice, related to the stability, and distance between H and its 1^{st} neighbors on the top layer of the surface.

Embodiment 20: The method of Embodiments 13-19, wherein the metal sulfide catalyst has a selectivity toward CO is 90% or greater, and wherein distinct CO2-reduction activity has a Faradaic efficiency of at least 35%.

### REFERENCES

1. Jones, J.-P., Prakash, G. K. S. & Olah, G. A. Electrochemical CO₂ Reduction: Recent Advances and Current Trends. Isr. J. Chem. 54, 1451-1466 (2014).
2. Yin, W. J. et al. The effect of excess electron and hole on CO₂ adsorption and activation on Rutile (110) surface. Sci. Rep. 6, 1-9 (2016).
3. Wu, J., Huang, Y., Ye, W. & Li, Y. CO₂ Reduction: From the Electrochemical to Photochemical Approach. Adv. Sci. 4, 1700194 (2017).
4. Kumar, B. et al. Photochemical and Photoelectrochemical Reduction of CO₂. Annu. Rev. Phys. Chem. 63, 541-569 (2012).
5. Li, Y. et al. Computational modeling of green hydrogen generation from photocatalytic H₂S splitting: Overview and perspectives. J. Photochem. Photobiol. C Photochem. Rev. 49, 100456 (2021). DOI 10.1016/j.jphotochemrev.2021.100456.
6. Vega, L. F. & Bahamon, D. Importance of Bridging Molecular and Process Modeling to Design Optimal Adsorbents for Large-Scale CO2 Capture. Acc. Chem. Res. 57, 188-197 (2024).
7. Norskov, J. K., Bligaard, T., Rossmeisl, J. & Christensen, C. H. Towards the computational design of solid catalysts. Nat. Chem. 1, 37-46 (2009).
8. Li, Y., Bahamon, D., Sinnokrot, M. & Vega, L. F. Computational screening of transition metal-doped CdS for photocatalytic hydrogen production. npj Comput. Mater. 8, 229 (2022).
9. Li, Y., Bahamon, D., Sinnokrot, M. & Vega, L. F. Insights into the mechanisms of H₂S adsorption and dissociation on CdS surfaces by DFT-D3 calculations. Int. J. Hydrogen Energy 48, 9700-9712 (2023).
10. Al Jitan, S. et al. Unprecedented photocatalytic conversion of gaseous and liquid CO₂ on graphene-impregnated Pt/Cu-TiO₂: The critical role of Cu dopant. J Environ. Chem. Eng. 11, 109485 (2023).
11. Borboudakis, G. et al. Chemically intuited, large-scale screening of MOFs by machine learning techniques. npj Comput. Mater. 3, 1-6 (2017).
12. Kumar, R. & Singh, A. K. Chemical hardness-driven interpretable machine learning approach for rapid search of photocatalysts. npj Comput. Mater. 7, 197 (2021).
13. Choubisa, H. et al. Interpretable discovery of new semiconductors with machine learning. npj Comput. Mater. 9, 117 (2023).
14. Esterhuizen, J. A., Goldsmith, B. R. & Linic, S. Interpretable machine learning for knowledge generation in heterogeneous catalysis. Nat. Catal. 5, 175-184 (2022).
15. Wang, S. H. et al. Infusing theory into deep learning for interpretable reactivity prediction. Nat. Commun. 12, 5288 (2021).
16. Jha, D. et al. Enhancing materials property prediction by leveraging computational and experimental data using deep transfer learning. Nat. Commun. 10, 5316 (2019).
17. Panapitiya, G. et al. Machine-Learning Prediction of CO Adsorption in Thiolated, Ag-Alloyed Au Nanoclusters. J. Am. Chem. Soc. 140, 17508-17514 (2018).
18. Wang, X. et al. Electric Dipole Descriptor for Machine Learning Prediction of Catalyst Surface-Molecular Adsorbate Interactions. J. Am. Chem. Soc. 142, 7737-7743 (2020).
19. Ulissi, Z. W., Medford, A. J., Bligaard, T. & Norskov, J. K. To address surface reaction network complexity using scaling relations machine learning and DFT calculations. Nat. Commun. 8, 14621 (2017).
20. Margraf, J. T., Jung, H., Scheurer, C. & Reuter, K. Exploring catalytic reaction networks with machine learning. Nat. Catal. 6, 112-121 (2023).
21. Mok, D. H. et al. Data-driven discovery of electrocatalysts for CO₂ reduction using active motifs-based machine learning. Nat. Commun. 14, 7303 (2023).
22. Zhu, X. et al. Activity Origin and Design Principles for Oxygen Reduction on Dual-Metal-Site Catalysts: A Combined Density Functional Theory and Machine Learning Study. J. Phys. Chem. Lett. 10, 7760-7766 (2019).
23. Zhang, N. et al. Machine Learning in Screening High Performance Electrocatalysts for CO₂ Reduction. Small Methods 5, 1-19 (2021).
24. Karthikeyan, M. Machine learning aided synthesis and screening of HER catalyst: Present developments and prospects. Catal. Rev.—Sci. Eng. 1-31 (2022).
25. Mazheika, A. et al. Artificial-intelligence-driven discovery of catalyst genes with application to CO₂ activation on semiconductor oxides. Nat. Commun. 13, 1-13 (2022).
26. Mou, T. et al. Bridging the complexity gap in computational heterogeneous catalysis with machine learning. Nat. Catal. 6, 122-136 (2023).
27. Jain, A. et al. Commentary: The materials project: A materials genome approach to accelerating materials innovation. APL Mater. 1, 11002 (2013).
28. Kirklin, S. et al. The Open Quantum Materials Database (OQMD): Assessing the accuracy of DFT formation energies. npj Comput. Mater. 1, 15010 (2015).
29. Winther, K. T. et al. Catalysis-hub.org: An Open Electronic Structure Database for Surface Reactions. 6, 75 (2019). DOI: 10.1038/s41597-019-0081-y.
30. Mamiyev, Z. & Balayeva, N. O. Metal Sulfide Photocatalysts for Hydrogen Generation: A Review of Recent Advances. Catalysts 12, 1-36 (2022).
31. El Aisnada, A. N., Miyauchi, M., Liu, M. & Yamaguchi, A. Recent update on electrochemical CO₂ reduction catalyzed by metal sulfide materials. Mater. Reports Energy 3, 100190 (2023).
32. Jia, L., Tan, X., Yu, T. & Ye, J. Mixed Metal Sulfides for the Application of Photocatalytic Energy Conversion. Energy & Fuels 36, 11308-11322 (2022).
33. Parsons, J. & Alotaibi, M. The Application of Transition Metal Sulfide Nanomaterials and Their Composite Nanomaterials in the Electrocatalytic Reduction of CO₂: A Review. Appl. Sci. 13, 3023 (2023).
34. Mukherjee, A., Abdinejad, M., Mahapatra, S. S. & Ruidas, B. C. Metal sulfide-based nanomaterials for electrochemical CO₂ reduction. J. Mater. Chem. A 11, 9300-9332 (2023).
35. Shen, W. et al. Applications of Rare Earth Promoted Transition Metal Sulfides in Electrocatalysis. Chinese J Chem. 41, 1740-1752 (2023).
36. Wang, J. et al. Nanostructured Metal Sulfides: Classification, Modification Strategy, and Solar-Driven CO₂ Reduction Application. Adv. Funct. Mater. 31, 1-39 (2021).
37. Benesty, J., Chen, J., Huang, Y. & Cohen, I. Pearson correlation coefficient. Springer Top. Signal Process. 2, 1-4 (2009).
38. Ouyang, R., Curtarolo, S., Ahmetcik, E., Scheffler, M. & Ghiringhelli, L. M. SISSO: A compressed-sensing method for identifying the best low-dimensional descriptor in an immensity of offered candidates. Phys. Rev. Mater. 2, 83802 (2018).
39. Wan, Z. & Wang, Q. De. Machine Learning Prediction of the Exfoliation Energies of Two-Dimension Materials via Data-Driven Approach. J. Phys. Chem. Lett. 12, 11470-11475 (2021).
40. Fung, V., Hu, G., Ganesh, P. & Sumpter, B. G. Machine learned features from density of states for accurate adsorption energy prediction. Nat. Commun. 12, 88 (2021).
41. Khan, M. M. 1—Introduction and fundamentals of chalcogenides and chalcogenides-based nanomaterials. Micro and Nano Technologies. 1-6 (2021).
42. Laio. Practical Electron Microscopy and Database, http://www.globalsino.com/EMI. (GlobalSino, 2006).
43. Jain, A. et al. The Materials Project: Accelerating Materials Design Through Theory-Driven Data and Tools. Handb. Mater. Model. 1-34 (2018).
44. Li, Z., Ma, X. & Xin, H. Feature engineering of machine-learning chemisorption models for catalyst design. Catal. Today 280, 232-238 (2017).
45. http://www.leonland.de/elements_by_price/list
46. Chandrasekaran, S. et al. Recent advances in metal sulfides: From controlled fabrication to electrocatalytic, photocatalytic and photoelectrochemical water splitting and beyond. Chem. Soc. Rev. 48, 4178-4280 (2019).
47. Shetty, V., Shedthi B, S. & Kumaraswamy, J. Predicting the thermodynamic stability of perovskite oxides using multiple machine learning techniques. Mater. Today Proc. 52, 457-461 (2022).
48. He, Y., Wang, Y., Zhang, L., Teng, B. & Fan, M. High-efficiency conversion of CO₂ to fuel over ZnO/g-C₃N₄ photocatalyst. Appl. Catal. B Environ. 168-169, 1-8 (2015).
49. Kumaravel, V., Mathew, S., Bartlett, J. & Pillai, S. C. Photocatalytic hydrogen production using metal doped TiO₂: A review of recent advances. Appl. Catal. B Environ. 244, 1021-1064 (2019).
50. Han, Z. Machine-learning-assisted design of a binary descriptor to decipher electronic and structural effects on sulfur reduction kinetics. Nat. Catal. 6, 1073-1086 (2023).
51. Jones, G., Bligaard, T., Abild-Pedersen, F. & Nørskov, J. K. Using scaling relations to understand trends in the catalytic activity of transition metals. J. Phys. Condens. Matter 20, 64239 (2008).
52. Cheng, J. et al. Bronsted-Evans-Polanyi relation of multistep reactions and volcano curve in heterogeneous catalysis. J Phys. Chem. C 112, 1308-1311 (2008).
53. Tran, K. & Ulissi, Z. W. Active learning across intermetallics to guide discovery of electrocatalysts for CO₂ reduction and H₂ evolution. Nat. Catal. 1, 696-703 (2018).
54. Wang, D. et al. Accelerated prediction of Cu-based single-atom alloy catalysts for CO₂ reduction by machine learning. Green Energy Environ. 8, 820-830 (2023).
55. Sun, G. et al. Performance of the Vienna ab initio simulation package (VASP) in chemical applications. J. Mol. Struct. THEOCHEM 624, 37-45 (2003).
56. Grimme, S., Antony, J., Ehrlich, S. & Krieg, H. A consistent and accurate ab initio parametrization of density functional dispersion correction (DFT-D) for the 94 elements H-Pu. J. Chem. Phys. 132, 154104 (2010).
57. Perdew, J. P., Burke, K. & Ernzerhof, M. Generalized gradient approximation made simple. Phys. Rev. Lett. 77, 3865-3868 (1996).
58. Blôchl, P. E. Projector augmented-wave method. Phys. Rev. B 50, 17953-17979 (1994).
59. Ong, S. P. et al. Python Materials Genomics (pymatgen): A robust, open-source python library for materials analysis. Comput. Mater. Sci. 68, 314-319 (2013).
60. Pedregosa, F. et al. Scikit-learn: Machine Learning in Python. J. Mach. Learn. Res. 12, 2825-2830 (2011).
61. g, Y., Hou, B., Jiang, H. & Zhang, J. Machine learning and artificial neural network accelerated computational discoveries in materials science. Wiley Interdiscip. Rev. Comput. Mol. Sci. 10, 1-21 (2020).
62. Sproviero, E. M., Gascón, J. A., McEvoy, J. P., Brudvig, G. W. & Batista, V. S. Quantum mechanics/molecular mechanics study of the catalytic cycle of water splitting in photosystem II. J. Am. Chem. Soc. 130, 3428-3442 (2008).
63. Awad, M. & Khanna, R. Support Vector Regression. in Efficient Learning Machines (eds. Awad, M. & Khanna, R.) 67-80 (Apress, 2015).
64. Deringer, V. L., Bartok, A. P., Bernstein, N., Wilkins, D. M., Ceriotti, M. & Csanyi, G. Gaussian Process Regression for Materials and Molecules. Chem. Rev. 121, 10073-10141 (2021).
65. Chen, Jiaqi, et al. “Photocatalytic Reduction of CO₂ by TiO₂ Modified Materials: Recent Advances and Outlook.” Energy & Fuels 38.9 (2024): 7614-7636.
66. Lu, Qingqing, et al. “Engineering graphitic carbon nitride (g-C₃N₄) for catalytic reduction of CO₂ to fuels and chemicals: Strategy and mechanism.” Green Chemistry 23.15 (2021): 5394-5428.
67. Li, Yuting, et al. “Computational screening of transition metal-doped CdS for photocatalytic hydrogen production.” npj Computational Materials 8.1 (2022): 229.
68. Woldu, Abebe Reda, et al. “Electrochemical CO₂ reduction (CO₂RR) to multi-carbon products over copper-based catalysts.” Coordination Chemistry Reviews 454 (2022): 214340.
69. Wei, Jumeng, et al. “Heterostructured electrocatalysts for hydrogen evolution reaction under alkaline conditions.” Nano-micro letters 10 (2018): 1-15.
70. Muhyuddin, Mohsin, et al. “Molybdenum disulfide as hydrogen evolution catalyst: From atomistic to materials structure and electrocatalytic performance.” Journal of Energy Chemistry (2023).
71. Mentel, L. M. mendeleev-A Python resource for properties of chemical elements, ions and isotopes. URL https//bitbucket. org/lukaszmentel/mendeleev (2014).
72. Ma, X. & Xin, H. Orbitalwise Coordination Number for Predicting Adsorption Properties of Metal Nanocatalysts. Phys. Rev. Lett. 118, 1-5 (2017).
73. Fung, V., Tao, F. F. & Jiang, D. E. General Structure-Reactivity Relationship for Oxygen on Transition-Metal Oxides. J. Phys. Chem. Lett. 8, 2206-2211 (2017).
74. Back, S. et al. Convolutional Neural Network of Atomic Surface Structures to Predict Binding Energies for High-Throughput Screening of Catalysts. J. Phys. Chem. Lett. 10, 4401-4408 (2019).
75. Gu, G. H. et al. Practical Deep-Learning Representation for Fast Heterogeneous Catalyst Screening. J. Phys. Chem. Lett. 11, 3185-3191 (2020).
76. Chen, Y., Huang, Y., Cheng, T. & Goddard, W. A. Identifying Active Sites for CO2 Reduction on Dealloyed Gold Surfaces by Combining Machine Learning with Multiscale Simulations. J. Am. Chem. Soc. 141, 11651-11657 (2019).
77. Chen, A., Zhang, X., Chen, L., Yao, S. & Zhou, Z. A Machine Learning Model on Simple Features for CO2 Reduction Electrocatalysts. J. Phys. Chem. C 124, 22471-22478 (2020).
78. Ma, X., Li, Z., Achenie, L. E. K. & Xin, H. Machine-Learning-Augmented Chemisorption Model for CO2 Electroreduction Catalyst Screening. J. Phys. Chem. Lett. 6, 3528-3533 (2015).
79. Noh, J., Back, S., Kim, J. & Jung, Y. Active learning with non-Ab initio input features toward efficient CO2 reduction catalysts. Chem. Sci. 9, 5152-5159 (2018).
80. Fung, V., Tao, F. F. & Jiang, D. E. Trends of Alkane Activation on Doped Cobalt (II, III) Oxide from First Principles. ChemCatChem 10, 244-249 (2018).
81. García-Muelas, R. & López, N. Statistical learning goes beyond the d-band model providing the thermochemistry of adsorbates on transition metals. Nat. Commun. 10, 4687 (2019).
82. Mamun, O., Winther, K. T., Boes, J. R. & Bligaard, T. A Bayesian framework for adsorption energy prediction on bimetallic alloy catalysts. npj Comput. Mater. 6, 1-11 (2020).
83. E. Bahadori et al., “High Pressure Photoreduction of CO2: Effect of Catalyst Formulation, Hole Scavenger Addition and Operating Conditions,” Catalysts, vol. 8, no. 10, 2018, doi: 10.3390/catal8100430.
84. I. Rossetti, A. Villa, C. Pirola, L. Prati, and G. Ramis, “A novel high-pressure photoreactor for CO2 photoconversion to fuels,” RSC Advances, vol. 4, no. 55, pp. 28883-28885, 2014, doi: 10.1039/C4RA03751K.
85. H. Jung et al., “Continuous-flow reactor with superior production rate and stability for CO2 reduction using semiconductor photocatalysts,” Energy & Environmental Science, vol. 16, no. 7, pp. 2869-2878, 2023, doi: 10.1039/D3EE00507K.
86. P. Franco, O. Sacco, V. Vaiano, and I. De Marco, “Supercritical Carbon Dioxide-Based Processes in Photocatalytic Applications,” Molecules, vol. 26, no. 9, 2021, doi: 10.3390/molecules26092640.
87. H. Kawanami, D. C. Grills, T. Ishizaka, M. Chatterjee, and A. Suzuki, “Photocatalytic reduction of CO2 under supercritical CO2 conditions: Effect of pressure, temperature, and solvent on catalytic efficiency,” Journal of CO2 Utilization, vol. 3-4, pp. 93-97, 2013, doi: https://doi.org/10.1016/j.jcou.2013.07.008.
88. G. Ebri, E. Alhashmi, Y. Baghdadi, M. Daboczi, S. Eslava, and K. Hellgardt, “Simultaneous photocatalytic CO2 reduction and C—C coupling of benzyl alcohol under high pressure and supercritical conditions,” Chemical Engineering Journal, vol. 505, p. 158356, 2025, doi: https://doi.org/10.1016/j.cej.2024.158356.

What is claimed is:

1. A method of converting CO2 to CO, the method comprising:
photocatalytically converting CO₂ to CO in the presence of a catalyst, wherein the catalyst comprises a metal sulfide catalyst, wherein the selectivity toward CO is 90% or greater, and wherein distinct CO2-reduction activity has a Faradaic efficiency of at least 35%.

2. The method of claim 1, wherein the metal sulfide catalyst comprises Ni₃S₂, MnS₂, VS₄, V₃S₄, or combinations thereof.

3. The method of claim 1, wherein the metal sulfide catalyst comprises MnS₂.

4. The method of claim 1, wherein the metal sulfide catalyst comprises V₃S₄.

5. The method of claim 1, wherein the method is conducted under a CO₂ pressure from 40 to 70 bar.

6. The method of claim 1, wherein the method is conducted for a period of time from 30 minutes to 48 hours.

7. The method of claim 1, wherein the method is conducted with 5 to 100 mg catalyst.

8. The method of claim 1, wherein the metal sulfide catalyst has a cathodic current density in an Ar-saturated blank (j_{Ar}) from −6.000 to −0.020 mA/cm².

9. The method of claim 1, wherein the metal sulfide catalyst has a cathodic current density in a CO₂-saturated blank (j_{CO2}) from −2.000 to −0.030 mA/cm².

10. The method of claim 1, wherein a difference (Δj) between a CO₂-saturated blank (j_{CO2}) a cathodic current density in an Ar-saturated blank (j_{Ar}) is from −0.020 to 4.000 mA/cm².

11. The method of claim 1, wherein the method is conducted using a graphite sheet modified with the metal sulfide catalyst.

12. The method of claim 1, wherein the method is conducted using an Ag/AgCl electrode.

13. A method for predicting CO chemisorption on a surface using metal sulfide catalysts, the method comprising:
a) inputting CO-surface distances, metal-sulfur bond lengths and energies above hull;
b) predicting the CO chemisorption on a surface; and
c) setting a minimum acceptable chemisorption on a surface; and
d) testing metal sulfide catalysts that meet the predicted minimum acceptable chemisorption.

14. The method of claim 13, wherein the predicting is conducted by applying machine learning algorithms.

15. The method of claim 14, wherein at least two machine learning algorithms are used.

16. The method of claim 14, wherein four machine learning algorithms are used.

17. The method of claim 14, wherein Random Forest, trained with data from Density Functional Theory is used.

18. The method of claim 13, wherein two physical inputs are also input.

19. The method of claim 18, wherein the two physical inputs are:
energy above the convex hull of bulk lattice, related to the stability, and distance between H and its 1st neighbors on the top layer of the surface.

20. The method of claim 13, wherein the metal sulfide catalyst has a selectivity toward CO is 90% or greater, and wherein distinct CO2-reduction activity has a Faradaic efficiency of at least 35%.
